(19) United States
(12) Patent Application Publication
(10) Pub. No.: US 2008/0206287 A1
(43) Pub. Date: Aug. 28, 2008

(54) Use of cyclosporin A to sensitize resistant cancer cells to death receptor ligands

(76) Inventors: John C. Reed, La Jolla, CA (US); Michael Paul Thomas, San Diego, CA (US)

Correspondence Address: Biotechnology Law Group;c/o Portfolioip, P.O. Box 52050, Minneapolis, MN, 55402, US

(21) Appl. No.: 11/901,582

(22) Filed: Sep. 17, 2007

Related U.S. Application Data
(60) Provisional application No. 60/845,716, filed on Sep. 18, 2006.

(51) Int. Cl. A61K 39/00; A61K 38/00; C12Q 1/04; C12N 5/06

(52) U.S. Cl. 424/277.1; 514/9; 435/375; 435/34

(57) ABSTRACT

Cyclosporin A has been discovered to sensitize cancers resistant to TNF-family death receptors such as TRAIL and Fas to ligand-mediated apoptosis. Therefore, compositions that include cyclosporin A are useful in treating such cancers.

### RELATED APPLICATIONS

This Application claims the benefit, under 35 U.S.C. § 119(e), of: U.S. Provisional Application No. 60/845,716, which was filed on Sep. 18, 2006, was of same title and named John C. Reed and Michael Paul Thomas, as inventors. The entirety of this application and document is incorporated by reference.

### STATEMENT OF GOVERNMENT RIGHTS

The invention was supported, at least in part, by a grant from the Government of the United States of America (grant no. CA78040 from the National Institutes of Health). The Government may have certain rights to the invention.

### BACKGROUND

Interest in tumor vaccines is increasing with recent announcements of promising clinical trials. Vaccine strategies partially rely on cytolytic T lymphocytes (CTLs) and natural killer (NK) cells to eliminate malignant cells by inducing rapid apoptosis. In part, these immune cells use death receptor ligands such as tumor necrosis factor-α (TNFα), FAS-L, and TRAIL to stimulate certain TNF-family death receptors on tumor target cells, resulting in activation of caspase-family proteases and triggering of apoptosis (Takeda et al., Nat. Med. 7:94-100, 2001; Montel et al., Cell Immunol. 165:312-317, 1995; Montel et al., Cell Immunol. 166:236-46, 1995; Sayers et al., J. Immunol. 161:3957-3965, 1998). Attempts to exploit these immune effector molecules as anticancer agents have resulted in early stage clinical trials that employ a recombinant soluble fragment of TRAIL and agonistic monoclonal antibodies targeting TRAIL receptors (Nagane et al., Apoptosis 6:191-197, 2001). A limitation of such therapies, however, is acquired or intrinsic resistance to TNF-family death ligands and death receptors, which commonly occurs in advanced malignancies (Wuchter et al., Leukemia 15:921-928, 2001).

TNF-family death receptors trigger apoptosis through a mechanism involving recruitment of certain caspase-family proteases to their cytosolic domains (e.g., caspases-8 and 10 in humans), resulting in formation of a death-inducing signaling complex (DISC). Upstream initiator caspases activated at the DISC then enter the cytosol, where they cleave and activate downstream effector caspases, resulting in apoptosis. This mechanism for achieving caspase activation is referred to as the “extrinsic” pathway, standing in contrast to another apoptosis pathway that involves mitochondria, and which has been termed the “intrinsic” pathway (Schimmer et al., Blood 98:3541-3553, 2001). Stimuli that activate the intrinsic pathway include DNA damaging anticancer drugs, γ-irradiation, hypoxia, and growth factor deprivation, causing mitochondria to release cytochrome c and other apoptogenic proteins into the cytosol, resulting in caspase activation (Hajra and Liu, Apoptosis 9:691-704, 2004).

Diverse mechanisms can create roadblocks to apoptosis within the extrinsic or intrinsic pathways, occurring commonly in many cancers during tumor progression and thus creating impediments to successful treatment. Documented resistance mechanisms relevant to the extrinsic pathway include reduced expression of TNF-family death receptors, shedding of soluble death receptors and expression of ligand-binding decoy receptors, reduced expression of caspases-8 and -10, and overexpression of intracellular caspase inhibitors (Wang and El-Deiry, Oncogene 22:8628-8633, 2003). Among the endogenous caspase inhibitors affecting the extrinsic pathway is c-FLIP, a protein resembling caspases-8 and -10, which can bind and prevent their activation at the DISC (Irmler et al., Nature 388:190-195, 1997; Scaffidi et al., J. Biol. Chem. 274:1541-1548, 1999).

Thus, there is a need for molecules that restore sensitivity of tumor cells to TNF-family death receptors and so are useful therapeutic adjuncts to anti-cancer agents such as recombinant TRAIL and tumor vaccines.

### SUMMARY OF THE INVENTION

We have discovered that cyclosporin A enhances the sensitivity of tumor cells that express TNF-family death receptors on the cell surface and are resistant to TNF-family death receptor to ligand-mediated apoptosis.

According to one embodiment of the invention, methods are provided for treating a cancer in a mammal in need of such treatment, such methods comprising administrating to the mammal an amount of a composition comprising cyclosporin A that is effective to increase killing of cells of the cancer mediated by a TNF-family death receptor ligand. Such methods are useful for enhancing the sensitivity of cancers that are resistant to killing mediated by such TNF-family death receptor ligands as, for example, TNF-α, Fas, lymphotoxin-α, TRAIL, DR3 ligand, DR6 ligand, NGF, and antagonistic antibody directed against TNFR1, FAS, DR3, DR4, DR5, DR6, or P75NTR. According to other embodiments of the invention, in addition to administration of a composition comprising cyclosporin A, a composition comprising additional substances is administered (whether co-administered or administered before or after the cyclosporin A-containing composition), including, but not limited to, one or more of the following: TNF-family death receptor ligand(s); compound(s) of formulas (I)-(VII) as described below, or anti-cancer agent(s); Alternatively, the composition comprising cyclosporin A may further comprise the TNF-family death receptor ligand(s), compound(s) of formulas (I)-(VII), anti-cancer agent(s), etc. Cancers that can be treated by the methods of the present invention include, but are not limited to, cancers of the lung, colon, breast, prostate, stomach, ovarian, liver, brain, skin, and blood, for example.

According to another embodiment of the invention, methods are provided for increasing killing of cells of a cancer that is resistant to killing mediated by a TNF-family death receptor ligand, such methods comprising contacting the cells ex vivo with an effective amount of a composition comprising cyclosporin A.

According to another embodiment of the invention, methods are provided for enhancing the efficacy of a vaccine against a cancer, such methods comprising administering to a mammal having a cancer an effective amount of the vaccine and administering an amount of a composition comprising cyclosporin A that is effective to increase killing of cells of the cancer. The vaccine and the composition comprising cyclosporin A may be co-administered, or the composition comprising cyclosporin A may be administered before or after the vaccine.

According to another embodiment of the invention, methods are provided for identifying an agent that enhances killing of cells of a cancer that is resistant to killing mediated by a TNF-family death receptor ligand, such methods comprising: (a) contacting a test sample of the cells under suitable conditions with: a composition comprising cyclosporin A; a composition comprising the agent; and a composition comprising the ligand; and (b) comparing death of the cells in the test sample to death of the cells in a control sample lacking the agent.

According to another aspect of the invention, compositions are provided for treating a cancer that is resistant to killing mediated by a TNF-family death receptor ligand, such compositions comprising the ligand and an amount of cyclosporin A that is effective to increase killing of the cancer mediated by the ligand.

According to another aspect of the invention, kits are provided for treating a cancer that is resistant to killing mediated by a TNF-family death receptor ligand, such kits comprising a composition comprising an amount of cyclosporin A that is effective to increase killing of the cancer mediated by the ligand, a composition comprising the ligand, and instructions for use. Alternatively, such kits may comprise a composition comprising the ligand and an amount of cyclosporin A that is effective to increase killing of the cancer mediated by the ligand, and instructions for use.

The foregoing and other aspects of the invention will become more apparent from the following detailed description, accompanying drawings, and the claims.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below.

### BRIEF DESCRIPTION OF THE FIGURES

FIG. 1 shows a summary of the results of screening of 2000 compounds using PPC-1 cells and agonistic Fas antibody (CH-11) for compounds that produced a reduction in cell viability of greater than or equal to 50 percent as determined by MTS assay. Of 89 hits, six compounds selectively sensitized PPC-1 cells to Fas at 2.5, 5, 10, and 25 μM and did not kill PPC-1 cells when applied without anti-Fas antibody. These six compounds were then tested for ability to sensitize other tumor cell lines besides PPC-1 to anti-Fas antibody. Of these six compounds, only cyclosporin A (CsA) displayed an ability to sensitize multiple tumor cell lines.

FIG. 2 shows that CsA sensitizes tumor cells to apoptosis induction by extrinsic pathway stimuli (agonistic anti-Fas antibody and TNF-family ligand TRAIL) but not intrinsic pathway stimuli VP16 (topoisomerase inhibitor) and Staurosporine (STS). PPC-1 prostate cancer cells (1×10⁴) were seeded overnight in 96-well plates. Cells were then treated overnight with varying concentrations of Cyclosporin A (0, 0.5, 1, 2, and 4 μM) in combination with varying concentrations of one of the following stimuli: Fas (0, 25, 50, 100, 200 ng/ml), TRAIL (0, 50, 100, 200, 400 ng/ml), VP-16 (0, 25, 50, 100, 200 μM) or STS (0, 0.25, 0.5, 1.0, and 2.0 nM). After 20-24 hours treatment, MTS was added and incubated for 4 hours. OD₄₉₀ was measured and cell viability numbers were calculated as percent of control values (mean ±SEM; n=3).

FIG. 3 shows results from a similar experiment performed using OVCAR-3 ovarian cancer cells, comparing the extrinsic pathway stimulus TRAIL with intrinsic pathway stimuli, VP16 and Paclitaxel (TAXOL) (microtubule aggregator). OVCAR-3 cells (1×10⁴) were seeded overnight in 96-well plates. Cells were treated varying concentrations of Cyclosporin A (0, 0.5, 1, 2, and 4 μM) in combination with varying concentrations of one of the following stimuli: TRAIL (0, 50, 100, 200, 400 ng/ml), VP-16 (0, 25, 50, 100, 200 uM) or TAXOL (0, 25, 0.50, 100, and 200 nM). Cell viability was determined by MTS assay one day later (mean ±SEM; n=3). Concentrations of CsA and TRAIL that resulted in synergistic induction of apoptosis are shown at the right side of the figure.

FIG. 4 shows examples of additional tumor cell lines where CsA sensitized to apoptosis induced by agonistic anti-Fas antibody CH11. Tumor cell lines included T47D and BT-549 breast cancers, HCT-15 colon cancer, IGROV-1 ovarian cancer, PPC1, ALVA-31, Du-145, and LNCaP prostate cancers, and U031, A498, 786-0, and RXF-393 renal cell cancer cell lines. Tumor cells were seeded overnight at 1×10⁴ to 5×10⁴ (based on cell type, size, and rate of growth) in RPMI, DMEM, McCoy's 5A MM, or Iscove's MEM containing 10% fetal bovine serum (FBS) with 2% Penicillin-Streptomycin in 96-well plates. Cells were then treated with increasing concentrations of Cyclosporin A (0 to 100 μM) in the presence or absence of agonistic Fas antibody (CH-11) at 100 ng/ml in the appropriate medium containing 2.5% FBS and antibiotics. After 20 to 24 hours, MTS was added and incubated for 4 hours. OD₄₉₀ measurements were taken and cell viability numbers were calculated as percent of control values.

FIG. 5 shows results from a similar experiment that was performed to examine the ability of CsA to sensitize various tumor cell lines to TRAIL, a cytokine member of the TNF family that also activates the extrinsic pathway for apoptosis. Tumor cell lines, including T47D, BT-549, MDA-MB-231, and MDA-MB-435 breast cancers, HT29, HCT116, HCT15, KM12, and COL0205 colon cancers, SK-OV3, OVCAR-3, and OVCAR-5 ovarian cancers, ALVA-31, PPC-1, PC3, and LNCaP prostate cancers, and A498, 786-0, and RXF-393 renal cell carcinoma cells, were seeded overnight at 1×10⁴ to 5×10⁴ (based on cell type, size, and rate of growth) in RPMI, DMEM, McCoy's SA MM, or Iscove's MEM containing 10% fetal bovine serum (FBS) with 2% Penicillin-Streptomycin in 96-well plates. Tumor cells were then treated with increasing concentrations of Cyclosporine A (0 to 100 μM) in the presence or absence of recombinant TRAIL (Apo2L) at 100 ng/ml in the appropriate medium containing 2.5% FBS and antibiotics. After 20 to 24 hours, MTS was added and incubated for 4 hours. OD₄₉₀ measurements were performed and percentage cell viability was calculated by comparison to control untreated cells.

FIG. 6 shows results of isobologram analysis that was performed to mathematically explore synergy of CsA with extrinsic pathway stimuli anti-FAS and TRAIL. Using data from PPC-1 cells, where checker-board titrations were performed at fixed molar ratios of CsA:Fas or CsA:TRAIL, isobologram analysis was performed using BioSyn software. The combination index (CI) was calculated with respect to the effective dose to achieve killing of 50%, 75%, or 90% of cells. CI values<0.3 indicate strong synergism.

### DETAILED DESCRIPTION OF THE INVENTION

The invention provides compositions, kits and methods for sensitizing TNF-family death receptor ligand-resistant cells to one or more TNF-family death receptor ligands using cyclosporin A, e.g., compositions and methods for sensitizing FAS ligand-resistant cancer cells to anti-FAS antibody-mediated killing. Thus, the invention provides compositions and methods for inducing apoptosis of cancer cells that are resistant to TNF-family death receptor ligand-mediated apoptosis. Cyclosporin A may alter extrinsic pathway apoptosis, or both extrinsic and intrinsic pathway apoptosis. In one embodiment, cyclosporin A alters extrinsic pathway apoptosis but does not alter FLIP expression in cells. In another embodiment, cyclosporin A alters extrinsic pathway apoptosis and alters FLIP expression.

The compositions of the invention may be employed to sensitize a cancer to any TNF-family death receptor ligand. Such TNF-family death receptor ligands include, but are not limited to, TNF-α, lymphotoxin-α, TRAIL, DR3 ligand, DR6 ligand, and NGF, as well as agonistic antibodies directed against TNFR1, FAS, DR3, DR4, DR5, DR6 or P75NTR. Such cancers include, but are not limited to, cancers of the lung, colon, breast, prostate, stomach, ovary, liver, brain, skin (e.g., melanoma), and blood (including, but not limited to, leukemia and lymphomas, and other forms of cancer, and other diseases of proliferation).

As described hereinbelow, cyclosporin A enhanced anti-FAS antibody-mediated killing of FAS ligand-resistant PPC-1 prostate cancer cells. PPC-1 cells are resistant to apoptosis induced by TRAIL and to agonistic antibodies targeting TNF-family death receptor FAS (CD95), despite expressing FAS and TRAIL receptors on their surface and expressing the requisite intracellular caspase activation machinery, including adaptor protein FADD and pro-caspases (Kim et al., J. Biol. Chem. 277:22320-22329, 2002). Cyclosporin A selectively sensitized PPC-1 cells to anti-FAS antibody, an extrinsic pathway agonist, without altering sensitivity to staurosporine and etoposide (VP16), which are intrinsic pathway agonists. Cyclosporin A did not increase FAS surface levels, and besides sensitizing PPC-1 cells to apoptosis induced by an agonistic anti-FAS antibody, also sensitized PPC-1 cells to apoptosis induced by TNF-family member TRAIL, consistent with a post-receptor mechanism. Cyclosporin A reduced expression of c-FLIP, an intracellular antagonist of the extrinsic pathway. Characterization of the effects of cyclosporin A on a panel of 27 cancer cell lines revealed that cyclosporin A generally sensitized resistant cells to extrinsic pathway agonists. Cyclosporin A is active in a variety of tumor cells and so is useful with TNF-family death receptor- or ligand-based anti-cancer therapies.

Cyclosporin A may be used to sensitize TNF-family death receptor ligand-resistant cells to one or more TNF-family death receptor ligands alone or in combination with other active ingredients. For example, cyclosporin A may be used in combination with one or more compounds of formula (I)-(8) described herein below. In addition, cyclosporin A may be used in combination with other known anti-cancer agents.

### DEFINITIONS

As used herein, “cyclosporin A” refers to cyclosporin A, cyclosporin A analogs, and any pharmaceutically acceptable salt thereof. A number of cyclosporin A analogs are known in the art. Since the original discovery of cyclosporin, a wide variety of naturally occurring cyclosporins have been isolated and identified and many further non-natural cyclosporins have been prepared by total- or semi-synthetic means or by the application of modified culture techniques. The class comprised by the cyclosporins is thus now substantial and includes, for example, the naturally occurring cyclosporins A through Z (see, for example, Traber et al., Helv. Chim. Acta 60:1247-1255, 1977; Traber et al., Helv. Chim. Acta 65:1655-1667, 1982; Kobel et al., Europ. J. Applied Microbiology and Biotechnology 14:237-240, 1982; and von Wartburg et al., Progress in Allergy 38:28-45, 1986), as well as various non-natural cyclosporin derivatives and artificial or synthetic cyclosporins including the dihydro- and iso-cyclosporins; derivatized cyclosporins (e.g., in which the 3′-O-atom of the -MeBmt-residue is acylated or a further substituent is introduced at the .alpha.-carbon atom of the sarcosyl residue at the 3-position); cyclosporins in which the -MeBmt-residue is present in isomeric form (e.g., in which the configuration across positions 6′ and 7′ of the -MeBmt-residue is cis rather than trans); and cyclosporins wherein variant amino acids are incorporated at specific positions within the peptide sequence employing, e.g., the total synthetic method for the production of cyclosporins developed by R. Wenger (see, for example, Traber et al., Helv. Chim. Acta 60:1247-1255, 1977; Traber et al., Helv. Chim. Acta 65:1655-1667, 1982; and Kobel et al., Europ. J. Applied Microbiology and Biotechnology 14:237-240, 1982; U.S. Pat. Nos. 4,108,985, 4,210,581, 4,220,641, 4,288,431, 4,554,351 and 4,396,542; European Patent Publications Nos. 0 034 567 and 0 056 782; International Patent Publication No. WO 86/02080; Wenger, Transpl. Proc. 15 [Suppl. 1] 2230, 1983; Wenger, Angew. Chem. Int. Ed., 24:77, 1985; and Wenger, Progress in the Chemistry of Organic Natural Products 50:123, 1986). Cyclosporin A analogues containing modified amino acids in the 1-position are reported by Rich et al., J. Med. Chem. 29:978, 1986. Immunosuppressive, anti-inflammatory, and anti-parasitic cyclosporin A analogues are described in U.S. Pat. Nos. 4,384,996; 4,771,122; 5,284,826; and 5,525,590, all assigned to Sandoz. Additional cyclosporin analogues are disclosed in WO 99/18120, assigned to Isotechnika. These and other cyclosporin A analogs may be used in the practice of the present invention.

The terms Ciclosporin, ciclosporin, cyclosporine, and Cyclosporine are interchangeable and refer to cyclosporin.

As used herein, “agent” refers to any substance that has a desired biological activity. An “anti-cancer agent” has detectable biological activity in treating cancer, e.g., in killing a cancer cell, treating or preventing cancer, reducing or stopping growth of a cancer, or reducing a symptom of a cancer, in a host.

As used herein, the statement “sensitize TNF-family death receptor ligand-resistant cells to one or more TNF-family death receptor ligands” refers to tumor cells that express TNF-family death receptors on the cell surface and are resistant to TNF-family death receptor ligand-mediated apoptosis. To “sensitize” such cells refers to treating the cells (1) an agonistic TNF-family death receptor ligand and (2) an amount of a composition comprising cyclosporin A that is effective to increase killing of the tumor cells relative to tumor cells contacted with the agonistic TNF-family death receptor ligand but not the composition comprising cyclosporin A.

As used herein, “effective amount” refers to an amount of a composition that causes a detectable difference in an observable biological effect, for example, a statistically significant difference in such an effect. The detectable difference may result from a single substance in the composition, from a combination of substances in the composition, or from the combined effects of administration of more than one composition. For example, an “effective amount” of a composition comprising cyclosporin A may refer to an amount of the composition that sensitizes TNF-family death receptor ligand-resistant cells to a TNF-family death receptor ligands, or another desired effect, e.g., to kill a cancer cell, to treat or prevent cancer or another disease or disorder, or to treat the symptoms of cancer or another disease or disorder, in a host. A combination of cyclosporin A and another substance, e.g., a compound of formula (I)-(VIII), an anti-cancer agent, or other active ingredient, in a given composition or treatment may be a synergistic combination. Synergy, as described for example by Chou and Talalay, Adv. Enzyme Regul. 22:27-55 (1984), occurs when the effect of the compounds when administered in combination is greater than the additive effect of the compounds when administered alone as a single agent. In general, a synergistic effect is most clearly demonstrated at suboptimal concentrations of the compounds. Synergy can be in terms of lower cytotoxicity, increased activity, or some other beneficial effect of the combination compared with the individual components.

As used herein, “treating” or “treat” includes (i) preventing a pathologic condition from occurring (e.g. prophylaxis); (ii) inhibiting the pathologic condition or arresting its development; (iii) relieving the pathologic condition; and/or diminishing symptoms associated with the pathologic condition.

As used herein, the term “patient” refers to organisms to be treated by the compositions and methods of the present invention. Such organisms include, but are not limited to, “mammals,” including, but not limited to, humans, monkeys, dogs, cats, horses, rats, mice, etc. In the context of the invention, the term “subject” generally refers to an individual who will receive or who has received treatment (e.g., administration of a compound of the invention, and optionally one or more anticancer agents) for cancer.

As used herein, “pharmaceutically acceptable salts” refer to derivatives of cyclosporin A or other disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The pharmaceutically acceptable salts of cyclosporin A or other compounds useful in the present invention can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., p. 1418 (1985), the disclosure of which is hereby incorporated by reference.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

One diastereomer of a compound disclosed herein may display superior activity compared with the other. When required, separation of the racemic material can be achieved by HPLC using a chiral column or by a resolution using a resolving agent such as camphonic chloride as in Thomas J. Tucker, et al., J. Med. Chem. 1994 37, 2437-2444. A chiral compound of Formula I may also be directly synthesized using a chiral catalyst or a chiral ligand, e.g. Mark A. Huffman, et al., J. Org. Chem. 1995, 60, 1590-1594.

“Stable compound” and “stable structure” are meant to indicate a compound that is sufficiently robust to survive isolation to a useful degree of purity from a reaction mixture, and formulation into an efficacious therapeutic agent. Only stable compounds are contemplated by the present invention.

“Substituted” is intended to indicate that one or more hydrogens on the atom indicated in the expression using “substituted” is replaced with a selection from the indicated group(s), provided that the indicated atom's normal valency is not exceeded, and that the substitution results in a stable compound. Suitable indicated groups include, e.g., alkyl, alkenyl, alkylidenyl, alkenylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and/or COOR^{x}, wherein each R^{x} and R^{y} are independently H, alkyl, alkenyl, aryl, heteroaryl, heterocycle, cycloalkyl or hydroxy. When a substituent is keto (i.e., ═O) or thioxo (i.e., ═S) group, then 2 hydrogens on the atom are replaced.

“Interrupted” is intended to indicate that in between two or more adjacent carbon atoms, and the hydrogen atoms to which they are attached (e.g., methyl (CH₃), methylene (CH₂) or methine (CH)), indicated in the expression using “interrupted” is inserted with a selection from the indicated group(s), provided that the each of the indicated atoms' normal valency is not exceeded, and that the interruption results in a stable compound. Such suitable indicated groups include, e.g., non-peroxide oxy (—O—), thio (—S—), carbonyl (—C(═O)—), carboxy (—C(═O)O—), imine (C═NH), sulfonyl (SO) or sulfoxide (SO₂).

Specific and preferred values listed below for radicals, substituents, and ranges, are for illustration only; they do not exclude other defined values or other values within defined ranges for the radicals and substituents “Alkyl” refers to a C₁-C₁₈ hydrocarbon containing normal, secondary, tertiary or cyclic carbon atoms. Examples are methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃.

The alkyl can optionally be substituted with one or more alkenyl, alkylidenyl, alkenylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and/or COOR^{x}, wherein each R^{x} and R^{y} are independently H, alkyl, alkenyl, aryl, heteroaryl, heterocycle, cycloalkyl or hydroxyl. The alkyl can optionally be interrupted with one or more non-peroxide oxy (—O—), thio (—S—), carbonyl (—C(═O)—), carboxy (—C(═O)O—), sulfonyl (SO) or sulfoxide (SO₂). Additionally, the alkyl can optionally be at least partially unsaturated, thereby providing an alkenyl.

“Alkenyl” refers to a C₂-C₁₈ hydrocarbon containing normal, secondary, tertiary or cyclic carbon atoms with at least one site of unsaturation, i.e. a carbon-carbon, sp2 double bond. Examples include, but are not limited to: ethylene or vinyl (—CH═CH₂), allyl (—CH₂CH═CH₂), cyclopentenyl (—C₅H₇), and 5-hexenyl (—CH₂ CH₂CH₂CH₂CH═CH₂).

The alkenyl can optionally be substituted with one or more alkyl, alkylidenyl, alkenylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and/or COOR^{x}, wherein each R^{x} and R^{y} are independently H, alkyl, alkenyl, aryl, heteroaryl, heterocycle, cycloalkyl or hydroxyl. Additionally, the alkenyl can optionally be interrupted with one or more non-peroxide oxy (—O—), thio (—S—), carbonyl (—C(═O)—), carboxy (—C(═O)O—), sulfonyl (SO) or sulfoxide (SO₂).

“Alkylidenyl” refers to a C₁-C₁₈ hydrocarbon containing normal, secondary, tertiary or cyclic carbon atoms. Examples are methylidenyl (═CH₂), ethylidenyl (═CHCH₃), 1-propylidenyl (═CHCH₂CH₃), 2-propylidenyl (═C(CH₃)₂), 1-butylidenyl (═CHCH₂CH₂CH₃), 2-methyl-1-propylidenyl (═CHCH(CH₃)₂), 2-butylidenyl (═C(CH₃)CH₂CH₃), 1-pentyl (═CHCH₂CH₂CH₂CH₃), 2-pentylidenyl (═C(CH₃)CH₂CH₂CH₃), 3-pentylidenyl (═C(CH₂CH₃)₂), 3-methyl-2-butylidenyl (═C(CH₃)CH(CH₃)₂), 3-methyl-1-butylidenyl (═CHCH₂CH(CH₃)₂), 2-methyl-1-butylidenyl (═CHCH(CH₃)CH₂CH₃), 1-hexylidenyl (═CHCH₂CH₂CH₂CH₂CH₃), 2-hexylidenyl (═C(CH₃)CH₂CH₂CH₂CH₃), 3-hexylidenyl (═C(CH₂CH₃)(CH₂CH₂CH₃)), 3-methyl-2-pentylidenyl (═C(CH₃)CH(CH₃)CH₂CH₃), 4-methyl-2-pentylidenyl (═C(CH₃)CH₂CH(CH₃)₂), 2-methyl-3-pentylidenyl (═C(CH₂CH₃)CH(CH₃)₂), and 3,3-dimethyl-2-butylidenyl (═C(CH₃)C(CH₃)₃.

The alkylidenyl can optionally be substituted with one or more alkyl, alkenyl, alkenylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and/or COOR^{x}, wherein each R^{x} and R^{y} are independently H, alkyl, alkenyl, aryl, heteroaryl, heterocycle, cycloalkyl or hydroxyl. Additionally, the alkylidenyl can optionally be interrupted with one or more non-peroxide oxy (—O—), thio (—S—), carbonyl (—C(═O)—), carboxy (—C(═O)O—), sulfonyl (SO) or sulfoxide (SO₂).

“Alkenylidenyl” refers to a C₂-C₁₈ hydrocarbon containing normal, secondary, tertiary or cyclic carbon atoms with at least one site of unsaturation, i.e. a carbon-carbon, sp² double bond. Examples include, but are not limited to: allylidenyl (═CHCH═CH₂), and 5-hexenylidenyl (═CHCH₂CH₂CH₂CH═CH₂).

The alkenylidenyl can optionally be substituted with one or more alkyl, alkenyl, alkylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and/or COOR^{x}, wherein each R^{x} and R^{y} are independently H, alkyl, alkenyl, aryl, heteroaryl, heterocycle, cycloalkyl or hydroxyl. Additionally, the alkenylidenyl can optionally be interrupted with one or more non-peroxide oxy (—O—), thio (—S—), carbonyl (—C(═O)—), carboxy (—C(═O)O—), sulfonyl (SO) or sulfoxide (SO₂).

“Alkylene” refers to a saturated, branched or straight chain or cyclic hydrocarbon radical of 1-18 carbon atoms, and having two monovalent radical centers derived by the removal of two hydrogen atoms from the same or different carbon atoms of a parent alkane. Typical alkylene radicals include, but are not limited to: methylene (—CH₂—) 1,2-ethyl (—CH₂CH₂—), 1,3-propyl (—CH₂CH₂CH₂—), 1,4-butyl (—CH₂CH₂CH₂CH₂—), and the like.

The alkylene can optionally be substituted with one or more alkyl, alkenyl, alkylidenyl, alkenylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and/or COOR^{x}, wherein each R^{x} and R^{y} are independently H, alkyl, alkenyl, aryl, heteroaryl, heterocycle, cycloalkyl or hydroxyl. Additionally, the alkylene can optionally be interrupted with one or more non-peroxide oxy (—O—), thio (—S—), carbonyl (—C(═O)—), carboxy (—C(═O)O—), sulfonyl (SO) or sulfoxide (SO₂). Moreover, the alkylene can optionally be at least partially unsaturated, thereby providing an alkenylene.

“Alkenylene” refers to an unsaturated, branched or straight chain or cyclic hydrocarbon radical of 2-18 carbon atoms, and having two monovalent radical centers derived by the removal of two hydrogen atoms from the same or two different carbon atoms of a parent alkene. Typical alkenylene radicals include, but are not limited to: 1,2-ethylene (—CH═CH—).

The alkenylene can optionally be substituted with one or more alkyl, alkenyl, alkylidenyl, alkenylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and/or COOR^{x}, wherein each R^{x} and R^{y} are independently H, alkyl, alkenyl, aryl, heteroaryl, heterocycle, cycloalkyl or hydroxyl. Additionally, The alkenylene can optionally be interrupted with one or more non-peroxide oxy (—O—), thio (—S—), carbonyl (—C(═O)—), carboxy (—C(═O)O—), sulfonyl (SO) or sulfoxide (SO₂).

The term “alkoxy” refers to the groups alkyl-O—, where alkyl is defined herein. Preferred alkoxy groups include, e.g., methoxy, ethoxy, n-propoxy, iso-propoxy, n-butoxy, tert-butoxy, sec-butoxy, n-pentoxy, n-hexoxy, 1,2-dimethylbutoxy, and the like.

The alkoxy can optionally be substituted with one or more alkyl, alkylidenyl, alkenylidenyl, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and COOR^{x}, wherein each R^{x} and R^{y} are independently H, alkyl, aryl, heteroaryl, heterocycle, cycloalkyl or hydroxyl.

The term “aryl” refers to an unsaturated aromatic carbocyclic group of from 6 to 20 carbon atoms having a single ring (e.g., phenyl) or multiple condensed (fused) rings, wherein at least one ring is aromatic (e.g., naphthyl, dihydrophenanthrenyl, fluorenyl, or anthryl). Preferred aryls include phenyl, naphthyl and the like.

The aryl can optionally be substituted with one or more alkyl, alkenyl, alkoxy, halo, haloalkyl, hydroxy, hydroxyalkyl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and COOR^{x}, wherein each R^{x} and R^{y} are independently H, alkyl, aryl, heteroaryl, heterocycle, cycloalkyl or hydroxyl.

The term “cycloalkyl” refers to cyclic alkyl groups of from 3 to 20 carbon atoms having a single cyclic ring or multiple condensed rings. Such cycloalkyl groups include, by way of example, single ring structures such as cyclopropyl, cyclobutyl, cyclopentyl, cyclooctyl, and the like, or multiple ring structures such as adamantanyl, and the like.

The cycloalkyl can optionally be substituted with one or more alkyl, alkenyl, alkoxy, halo, haloalkyl, hydroxy, hydroxyalkyl, aryl, heteroaryl, heterocycle, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and COOR^{x}, wherein each R^{x} and R^{y} are independently H, alkyl, aryl, heteroaryl, heterocycle, cycloalkyl or hydroxyl.

The cycloalkyl can optionally be at least partially unsaturated, thereby providing a cycloalkenyl.

The term “halo” refers to fluoro, chloro, bromo, and iodo. Similarly, the term “halogen” refers to fluorine, chlorine, bromine, and iodine.

“Haloalkyl” refers to alkyl as defined herein substituted by 1-4 halo groups as defined herein, which may be the same or different. Representative haloalkyl groups include, by way of example, trifluoromethyl, 3-fluorododecyl, 12,12,12-trifluorododecyl, 2-bromooctyl, 3-bromo-6-chloroheptyl, and the like.

The term “heteroaryl” is defined herein as a monocyclic, bicyclic, or tricyclic ring system containing one, two, or three aromatic rings and containing at least one nitrogen, oxygen, or sulfur atom in an aromatic ring, and which can be unsubstituted or substituted, for example, with one or more, and in particular one to three, substituents, like halo, alkyl, hydroxy, hydroxyalkyl, alkoxy, alkoxyalkyl, haloalkyl, nitro, amino, alkylamino, acylamino, alkylthio, alkylsulfinyl, and alkylsulfonyl. Examples of heteroaryl groups include, but are not limited to, 2H-pyrrolyl, 3H-indolyl, 4H-quinolizinyl, 4nH-carbazolyl, acridinyl, benzo[b]thienyl, benzothiazolyl, P3-carbolinyl, carbazolyl, chromenyl, cinnaolinyl, dibenzo[b,d]furanyl, furazanyl, furyl, imidazolyl, imidizolyl, indazolyl, indolisinyl, indolyl, isobenzofuranyl, isoindolyl, isoquinolyl, isothiazolyl, isoxazolyl, naphthyridinyl, naptho[2,3-b], oxazolyl, perimidinyl, phenanthridinyl, phenanthrolinyl, phenarsazinyl, phenazinyl, phenothiazinyl, phenoxathiinyl, phenoxazinyl, phthalazinyl, pteridinyl, purinyl, pyranyl, pyrazinyl, pyrazolyl, pyridazinyl, pyridyl, pyrimidinyl, pyrimidinyl, pyrrolyl, quinazolinyl, quinolyl, quinoxalinyl, thiadiazolyl, thianthrenyl, thiazolyl, thienyl, triazolyl, and xanthenyl. In one embodiment the term “heteroaryl” denotes a monocyclic aromatic ring containing five or six ring atoms containing carbon and 1, 2, 3, or 4 heteroatoms independently selected from the group non-peroxide oxygen, sulfur, and N(Z) wherein Z is absent or is H, O, alkyl, phenyl or benzyl. In another embodiment heteroaryl denotes an ortho-fused bicyclic heterocycle of about eight to ten ring atoms derived therefrom, particularly a benz-derivative or one derived by fusing a propylene, or tetramethylene diradical thereto.

The heteroaryl can optionally be substituted with one or more alkyl, alkenyl, alkoxy, halo, haloalkyl, hydroxy, hydroxyalkyl, aryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and COOR^{x}, wherein each R^{x} and R^{y} are independently H, alkyl, aryl, heteroaryl, heterocycle, cycloalkyl or hydroxyl.

The term “heterocycle” refers to a saturated or partially unsaturated ring system, containing at least one heteroatom selected from the group oxygen, nitrogen, and sulfur, and optionally substituted with alkyl or C(═O)OR^{b}, wherein R^{b} is hydrogen or alkyl. Typically heterocycle is a monocyclic, bicyclic, or tricyclic group containing one or more heteroatoms selected from the group oxygen, nitrogen, and sulfur. A heterocycle group also can contain an oxo group (═O) attached to the ring. Non-limiting examples of heterocycle groups include 1,3-dihydrobenzofuran, 1,3-dioxolane, 1,4-dioxane, 1,4-dithiane, 2H-pyran, 2-pyrazoline, 4H-pyran, chromanyl, imidazolidinyl, imidazolinyl, indolinyl, isochromanyl, isoindolinyl, morpholine, piperazinyl, piperidine, piperidyl, pyrazolidine, pyrazolidinyl, pyrazolinyl, pyrrolidine, pyrroline, quinuclidine, and thiomorpholine.

The heterocycle can optionally be substituted with one or more alkyl, alkenyl, alkoxy, halo, haloalkyl, hydroxy, hydroxyalkyl, aryl, heteroaryl, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and COOR^{x}, wherein each R^{x} and R^{y} are independently H, alkyl, aryl, heteroaryl, heterocycle, cycloalkyl or hydroxyl.

Examples of nitrogen heterocycles and heteroaryls include, but are not limited to, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, morpholino, piperidinyl, tetrahydrofuranyl, and the like as well as N-alkoxy-nitrogen containing heterocycles. In one specific embodiment of the invention, the nitrogen heterocycle can be 3-methyl-5,6-dihydro-4H-pyrazino[3,2,1-jk]carbazol-3-ium iodide.

Another class of heterocyclics is known as “crown compounds” which refers to a specific class of heterocyclic compounds having one or more repeating units of the formula [—(CH₂—)ₐA-] where a is equal to or greater than 2, and A at each separate occurrence can be O, N, S or P. Examples of crown compounds include, by way of example only, [—(CH₂)₃—NH—]₃, [—((CH₂)₂—O)₄—((CH₂)₂—NH)₂] and the like. Typically such crown compounds can have from 4 to 10 heteroatoms and 8 to 40 carbon atoms.

The term “alkanoyl” refers to C(═O)R, wherein R is an alkyl group as previously defined.

The term “acyloxy” refers to —O—C(═O)R, wherein R is an alkyl group as previously defined. Examples of acyloxy groups include, but are not limited to, acetoxy, propanoyloxy, butanoyloxy, and pentanoyloxy. Any alkyl group as defined above can be used to form an acyloxy group.

The term “alkoxycarbonyl” refers to C(═O)OR, wherein R is an alkyl group as previously defined.

The term “amino” refers to —NH₂, and the term “alkylamino” refers to —NR₂, wherein at least one R is alkyl and the second R is alkyl or hydrogen. The term “acylamino” refers to RC(═O)N, wherein R is alkyl or aryl.

The term “imino” refers to —C═NH.

The term “nitro” refers to —NO₂.

The term “trifluoromethyl” refers to —CF₃.

The term “trifluoromethoxy” refers to —OCF₃.

The term “cyano” refers to —CN.

The term “hydroxy” or “hydroxyl” refers to —OH.

The term “oxy” refers to —O—.

The term “thio” refers to —S—.

The term “thioxo” refers to (═S).

The term “keto” refers to (═O).

As to any of the above groups, which contain one or more substituents, it is understood, of course, that such groups do not contain any substitution or substitution patterns which are sterically impractical and/or synthetically non-feasible. In addition, the compounds of this invention include all stereochemical isomers arising from the substitution of these compounds.

Selected substituents within the compounds described herein are present to a recursive degree. In this context, “recursive substituent” means that a substituent may recite another instance of itself. Because of the recursive nature of such substituents, theoretically, a large number may be present in any given claim. One of ordinary skill in the art of medicinal chemistry understands that the total number of such substituents is reasonably limited by the desired properties of the compound intended. Such properties include, by of example and not limitation, physical properties such as molecular weight, solubility or log P, application properties such as activity against the intended target, and practical properties such as ease of synthesis.

Recursive substituents are an intended aspect of the invention. One of ordinary skill in the art of medicinal and organic chemistry understands the versatility of such substituents. To the degree that recursive substituents are present in an claim of the invention, the total number will be determined as set forth above.

The compounds described herein can be administered as the parent compound, a pro-drug of the parent compound, or an active metabolite of the parent compound.

“Pro-drugs” are intended to include any covalently bonded substances which release the active parent drug or other formulas or compounds of the present invention in vivo when such pro-drug is administered to a mammalian subject. Pro-drugs of a compound of the present invention are prepared by modifying functional groups present in the compound in such a way that the modifications are cleaved, either in routine manipulation in vivo, to the parent compound. Pro-drugs include compounds of the present invention wherein the carbonyl, carboxylic acid, hydroxy or amino group is bonded to any group that, when the pro-drug is administered to a mammalian subject, cleaves to form a free carbonyl, carboxylic acid, hydroxy or amino group. Examples of pro-drugs include, but are not limited to, acetate, formate and benzoate derivatives of alcohol and amine functional groups in the compounds of the present invention, and the like.

“Metabolite” refers to any substance resulting from biochemical processes by which living cells interact with the active parent drug or other formulas or compounds of the present invention in vivo, when such active parent drug or other formulas or compounds of the present are administered to a mammalian subject. Metabolites include products or intermediates from any metabolic pathway.

“Metabolic pathway” refers to a sequence of enzyme-mediated reactions that transform one compound to another and provide intermediates and energy for cellular functions. The metabolic pathway can be linear or cyclic.

Obviously, numerous modifications and variations of the present invention are possible in light of the above teachings. It is therefore to be understood that within the scope of the appended claims, the invention may be practiced otherwise than as specifically described herein.

A specific group of compounds of the invention have the formula (I):

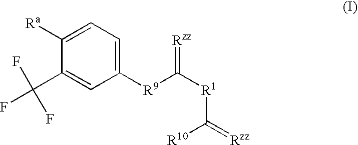

wherein,

R¹ is alkylene, alkenylene, arylene, heteroarylene, heterocyclene or cycloalkylene;

R^{a} is F, Cl, Br or I;

R⁹ is O or NR^{x};

each R^{zz} is independently O, NR^{x} or S;

R¹⁰ is alkyl, alkenyl, alkoxy, halo, haloalkyl, hydroxy, hydroxyalkyl, heteroaryl, heterocycle, cycloalkyl, amino, alkylamino, NR^{x}R^{y} or COOR^{x}; and

each R^{x} and R^{y} is independently H, alkyl, alkenyl, aryl, heteroaryl, heterocycle, cycloalkyl or hydroxyl;

or a pharmaceutically acceptable salt thereof.

A specific group of compounds of the invention have the formula (II):

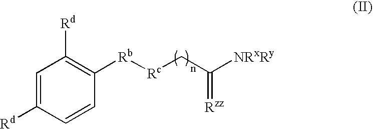

wherein,

n is 0-5;

R^{b} is O, S or NR^{z}, wherein R^{z} is H or alkyl;

R^{c} is alkylene, alkenylene, arylene, heteroarylene, heterocyclene or cycloalkylene;

each R^{d} is independently halo, haloalkyl, hydroxyl, hydroxyalkyl, nitro, trifluoromethyl, trifluoromethoxy, cyano, or COOH;

R^{zz} is O or NH;

R^{x} and R^{y} are each independently H, alkyl, alkenyl, aryl, heteroaryl, heterocycle, cycloalkyl or hydroxyl;

or a pharmaceutically acceptable salt thereof.

A specific group of compounds of the invention have the formula (III):

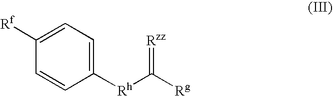

wherein,

R^{f} is halo, haloalkyl, hydroxyl, hydroxyalkyl, nitro, trifluoromethyl, trifluoromethoxy, cyano, or COOH;

R^{g} is CRⁱR^{j} or NRⁱR^{j}, wherein each Rⁱ and R^{j} is independently alkyl, alkenyl, alkoxy, haloalkyl, hydroxy, hydroxyalkyl, aryl, heteroaryl, heterocycle, cycloalkyl, or Rⁱ and R^{j} together form a heteroaryl or heterocycle optionally substituted with an arylalkenylene, arylalkylene, heteroarylalkylene, or heteroarylalkenylene;

R^{h} is alkylene or alkenylene; and

R^{zz} is O, NH or S;

or a pharmaceutically acceptable salt thereof.

A specific group of compounds of the invention have the formula (IV):

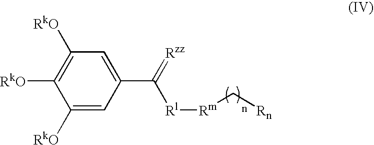

wherein,

n is 1-4;

each R^{k} is independently H, alkyl or alkenyl;

R¹ is O, alkylene, alkenylene or NR^{z}, wherein R^{z} is H or alkyl;

R^{m} is O, alkylene, alkenylene or NR^{z}, wherein R^{z} is H or alkyl;

Rⁿ is alkyl, alkenyl, alkoxy, haloalkyl, hydroxyalkyl, aryl, heteroaryl, heterocycle, or cycloalkyl; and

R^{zz} is O, NH or S;

or a pharmaceutically acceptable salt thereof.

A specific group of compounds of the invention have the formula (V):

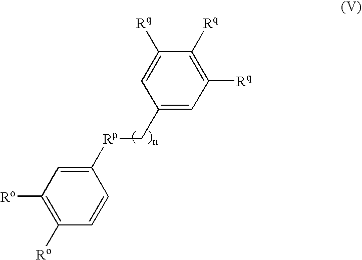

wherein,

n is 1-4;

each R^{o} is independently H, alkyl, alkoxy or alkenyl;

R^{p} is O or NR^{z}, wherein R^{z} is H or alkyl;

each R^{q} is independently alkoxy or trifluoromethoxy;

or a pharmaceutically acceptable salt thereof.

A specific group of compounds of the invention have the formula (VI):

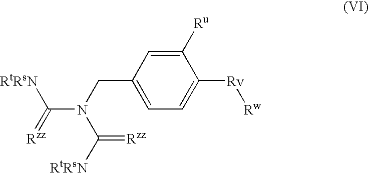

wherein,

each R^{s} is independently H or alkyl;

each R^{t} is independently H or alkyl;

R^{u} is halo, haloalkyl, hydroxyl, hydroxyalkyl, nitro, trifluoromethyl, trifluoromethoxy, cyano, or COOH;

R^{v} is O Or NR^{z};

R^{z} is H or alkyl;

R^{w} is alkyl, alkenyl, alkoxy, haloalkyl, hydroxyalkyl, aryl, heteroaryl, heterocycle, or cycloalkyl; and

each R^{zz} is independently O, NH or S;

or a pharmaceutically acceptable salt thereof.

A specific group of compounds of the invention have the formula (VII):

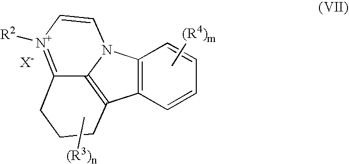

wherein,

R² is alkyl or alkenyl;

each R³ is independently H, alkyl, alkylidenyl, alkenylidenyl, alkoxy, halo, haloalkyl, hydroxy, hydroxyalkyl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, imino, keto, thioxo, NR^{x}R^{y} or COOR^{x};

each R⁴ is independently H, alkyl, alkoxy, halo, haloalkyl, hydroxy, hydroxyalkyl, heteroaryl, heterocycle, cycloalkyl, alkanoyl, alkoxycarbonyl, amino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} or COOR^{x};

each R^{x} and R^{y} are independently H, alkyl, aryl, heteroaryl, heterocycle, cycloalkyl or hydroxyl;

n is 0-3;

m is 0-5; and

X is a suitable counterion;

or a pharmaceutically acceptable salt thereof.

A specific group of compounds of the invention have the formula (VIII):

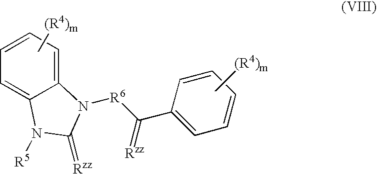

wherein,

each R⁴ is independently H, alkyl, alkoxy, halo, haloalkyl, hydroxy, hydroxyalkyl, heteroaryl, heterocycle, cycloalkyl, alkanoyl, alkoxycarbonyl, amino, alkylamino, acylamino, nitro, trifluoromethyl, trifluoromethoxy, carboxy, carboxyalkyl, alkylthio, alkylsulfinyl, alkylsulfonyl, cyano, NR^{x}R^{y} and COOR^{x};

each R^{x} and R^{y} are independently H, alkyl, aryl, heteroaryl, heterocycle, cycloalkyl or hydroxyl;

R⁵ is H, alkyl, arylalkyl, or alkenyl;

R⁶ is alkylene or alkenylene;

each R^{zz} is independently O, NR^{x} or S; and

each m is independently 0-5;

or a pharmaceutically acceptable salt thereof.

### Specific Ranges, Values, and Embodiments

Specific ranges, values, and embodiments provided below are for illustration purposes only and do not otherwise limit the scope of the invention, as defined by the claims.

For the compound of formula (I):

A specific value for R¹ is alkylene.

Another specific value for R¹ is methylene.

A specific value for R^{a} is Cl.

A specific value for R⁹ is NR^{x}.

Another specific value for R⁹ is NH.

Another specific value for each R^{zz} is O.

A specific value for R^{x} is H.

A specific value for R¹⁰ is alkyl.

Another specific value for R¹⁰ is methyl.

For the compound of formula (II):

A specific value for n is 0.

A specific value for R^{b} is O.

A specific value for R^{c} is alkylene.

Another specific value for R^{c} is n-propylene.

A specific value for each R^{d} is halo.

Another specific value for each R^{d} is chloro.

A specific value for R^{zz} is O.

A specific value for R^{x} is H.

A specific value for R^{y} is hydroxyl.

or a pharmaceutically acceptable salt thereof.

For the compound of formula (III):

A specific value for R^{f} is halo.

Another specific value for R^{f} is chloro.

A specific value for R^{g} is NRⁱR^{j}, wherein Rⁱ and R^{j} together form a heterocycle, substituted with an arylalkenylene, arylalkylene, heteroarylalkylene, or heteroarylalkenylene.

Another specific value for R^{g} is NRⁱR^{j}, wherein Rⁱ and R^{j} together form a heterocycle, substituted with an arylalkenylene.

A specific value for R^{h} is alkylene.

Another specific value for R^{h} is methylene.

A specific value for R^{zz} is O.

For the compound of formula (IV):

A specific value for n is 1.

A specific value for each R^{k} is alkyl.

Another specific value for each R^{k} is methyl.

A specific value for R¹ is NR^{z}, wherein R^{z} is H.

A specific value for R^{m} is NR^{z}, wherein R^{z} is H.

A specific value for Rⁿ is heterocycle.

A specific value for R^{zz} is O.

For the compound of formula (V):

A specific value for n is 1.

A specific value for each R^{o} is alkyl.

Another specific value for each R^{o} is methyl.

A specific value for R^{o} is NR^{z}, wherein R^{z} is H.

A specific value for each R^{q} is alkoxy.

For the compound of formula (VI):

A specific value for each R^{s} is H.

A specific value for each R^{t} is H.

A specific value for R^{u} is halo.

Another specific value for R^{u} is chloro.

A specific value for R^{v} is O.

A specific value for R^{w} is alkyl.

Another specific value for R^{w} is sec-butyl.

A specific value for each R^{zz} is NH.

For the compound of formula (VII):

A specific value for R² is alkyl.

Another specific value for R² is methyl.

A specific value for each R³ is H.

A specific value for each R⁴ is cycloalkyl.

A specific value for each R⁴ is cyclohexyl.

A specific value for n is 1.

A specific value for m is 1.

A specific value for X is iodo.

For the compound of formula (VIII):

A specific value for each R⁴ is independently H, alkyl or hydroxyl.

Another specific value for each R⁴ is independently H, tert-butyl or hydroxyl.

A specific value for R⁵ is alkyl.

Another specific value for R⁵ is alkyl, which is optionally substituted with aryl, which is optionally substituted with alkyl.

Another specific value for R⁵ is alkyl, which is optionally substituted with aryl.

Another specific value for R⁵ is alkyl, which is optionally substituted with cycloalkyl.

Another specific value for R⁵ is methyl.

Another specific value for R⁵ is ethyl.

Another specific value for R⁵ is 1-ethylpiperidinyl.

Another specific value for R⁵ is 4-methyl benzyl.

Another specific value for R⁵ is benzyl.

A specific value for R⁶ is alkylene.

Another specific value for R⁶ is methylene.

A specific value for each R^{zz} is independently O or NR^{x}.

A specific value for each R^{zz} is independently O or NR^{x}, wherein R^{x} is H.

A specific value for each m is 1.

Another specific value for m is 2.

Another specific value for m is 3.

The compositions of the invention can be formulated as pharmaceutical compositions and administered to a mammalian host, such as a human patient, in a variety of forms adapted to the chosen route of administration, i.e., orally or parenterally, by intravenous, intramuscular, topical or subcutaneous routes.

Such compositions may be systemically administered in vivo by a variety of routes. For example, they may be administered orally, in combination with a pharmaceutically acceptable excipients such as an inert diluent or an assimilable edible carrier. They may be enclosed in hard or soft shell gelatin capsules, may be compressed into tablets, or may be incorporated directly with the food of the patient's diet. For oral administration, the active ingredient or ingredients may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active ingredient in such useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The compositions may also be administered intravenously or intraperitoneally by infusion or injection. Solutions of the cyclosporin, its salts and other active ingredients can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating cyclosporin A or other active ingredients in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, cyclosporin A and other active ingredients may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of cyclosporin A or other active ingredients can be determined by comparing their in vitro activity and in vivo activity in animal models. Methods for the extrapolation of effective dosages in mice, and other animals, to humans are known to the art; for example, see U.S. Pat. No. 4,938,949.

Generally, the concentration of cyclosporin or other active ingredients of the invention in a liquid composition, such as a lotion, will be from about 0.1-25 wt-%, preferably from about 0.5-10 wt-%. The concentration in a semi-solid or solid composition such as a gel or a powder will be about 0.1-5 wt-%, preferably about 0.5-2.5 wt-%.

The amount of the compound, or an active salt or derivative thereof, required for use alone or with other anticancer compounds will vary not only with the particular salt selected but also with the route of administration, the nature of the condition being treated and the age and condition of the patient and will be ultimately at the discretion of the attendant physician or clinician.

In general, however, a suitable dose may be in the range of from about 0.5 to about 100 mg/kg, e.g., from about 1 to about 75 mg/kg of body weight per day, or 1.5 to about 50 mg per kilogram body weight of the recipient per day, or about 2 to about 30 mg/kg/day, or about 2.5 to about 15 mg/kg/day.

The compound may be conveniently administered in unit dosage form; for example, containing 5 to 1000 mg, conveniently 10 to 750 mg, most conveniently, 50 to 500 mg of active ingredient per unit dosage form.

The active ingredient may be administered to achieve peak plasma concentrations of the active compound of from about 0.5 to about 75 μM, preferably, about 1 to 50 μM, most preferably, about 2 to about 30 mM. This may be achieved, for example, by the intravenous injection of a 0.05 to 5% solution of the active ingredient, optionally in saline, or orally administered as a bolus containing about 1-100 mg of the active ingredient. Desirable blood levels may be maintained by continuous infusion to provide about 0.01-5.0 mg/kg/hr or by intermittent infusions containing about 0.4-15 mg/kg of the active ingredient(s).

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

A patient may also be treated by ex vivo administration of compositions according to the present invention according to known protocols. Cells of the patient that include TNF-family death receptor ligand-resistant cancer cells are removed from the patient, treated under suitable conditions with cyclosporin A, an appropriate TNF-family death receptor ligand, and other agents as desired to kill the cancer cells, and returned to the patient's body.

The following invention will be further described by the following nonlimiting example.

### EXAMPLE 1

### Experimental Methods

Reagents. A 50,000 compound Diversa chemical library was obtained from Chembridge (San Diego, Calif.). The anti-FAS monoclonal antibody CH-11 was purchased from MBL (MBL, Co. Ltd., Nagoya, Japan). TRAIL was obtained from Alexis (San Diego, Calif.). VP-16 and staurosporine were purchased from Sigma (Sigma Inc., Milwaukee, Wis.). 2-cyano-3,12-dioxooleana-1,9-dien-28-oic acid (CDDO) was a generous gift from Michael Sporn (Dartmouth University).

Cell Lines. Cell lines were maintained in RPMI 1640 supplemented with 2.5-10% fetal calf serum (FCS) (Hyclone, Tulare, Calif.), 1 mM L-glutamine and antibiotics (streptomycin/penicillin). Cells were cultured at 37° C. in a humid atmosphere with 5% CO₂.

High throughput screening. Screens were performed using a fully integrated, programmable robotic liquid handling system (Biomek® FX, Beckman-Coulter Inc., Fullerton, Calif.), with integrated plate reader (LJL analyst HT 96-384, Sunnyvale, Calif.) and environmentally controlled plate carousel set at 37° C. and 5% CO₂:95% air. PPC-1 cells (1×10⁴) were seeded overnight into 96 well, flat-bottom plates (Costar, Cambridge, Mass.) in 100 μL of medium containing 2.5% FCS. The next day, aliquots from the 50,000 compound Diversa library were added at a final concentration of 7.5 μg/mL (about 25 μM) in a final concentration of 0.5% (v:v) dimethylsulfoxide (DMSO). CH-11 antibody (100 ng/mL) was then added, and the cells were incubated for 24 hours before assessing cell viability by a 3-(4,5-Dimethylthiazol-2-yl)-2,5-diphenyltetrazolium Bromide (MTT) dye reduction assay (Sigma).

Cell death assays. Cell viability was measured by MTT and MTS assays, essentially as described in Schimmer et al. (Cancer Cell 5:25-35, 2004). Absorbance readings were plotted against a standard curve to derive the corresponding cell number, and cell viability was expressed as a percentage relative to untreated cells. Apoptosis was measured by flow cytometric analysis of Annexin V surface expression after staining cells with FITC-anti-Annexin V and propidium iodide (PI) (Biovision, Mountain View, Calif.), as described in Pedersen et al. (Blood 100:2965-72, 2002).

Cell transfections. PPC-1 cells (2×10⁵) were seeded in 35 mm diameter plates in RPMI with 10% FCS. The next day the cells were co-transfected using Lipofectamine Plus (Invitrogen, Carlsbad, Calif.) with 0.5 μg GFP-encoding plasmid pEGFP (Invitrogen) in combination with 1.5 Hg of plasmids encoding Bcl-XL, the viral caspase-8 inhibitor Crm A, or empty vector. At 2 days post-transfection, cells were incubated with various concentrations of CH-11 antibody and the test compounds for 24 hours, then the percentage apoptosis was scored by UV-microscopic analysis of the GFP-positive cells, counting a minimum of 200 cells. Cells that had rounded up and were floating in the medium were counted as non-viable, while cells that remained adherent to the plate with normal morphological features were counted as viable.

Immunoblot analysis. Protein extracts were obtained by washing cells with phosphate-buffered saline (PBS) [pH 7.4] and suspending the cells in lysis buffer [10 mM Tris (pH 7.4), 150 mM NaCl, 0.1% Triton X-100, 0.5% sodium deoxycholate, and 5 mM EDTA] containing protease inhibitors (Complete tablets; Roche, Indianapolis, Ind.). Immunoblot assays were performed as described in Carter et al. (Blood 105:4043-4050, 2005). Briefly, equal amounts of protein as determined by a Bradford assay (Bradford, Anal. Biochem. 72:248-54, 1976) were subjected to SDS-PAGE (4-20% gradient gels from ISC BioExpress, Kaysville, Utah), followed by transfer to nitrocellulose membranes. Membranes were incubated with mouse monoclonal anti-human FLIP (NF6 clone) (1:500 v/v) (Alexis, San Diego, Calif.), 1:1,000 (v/v) anti-aspase-8 clone 5F7 (Upstate) and mouse monoclonal anti-tubulin (1:2000 v/v) (Sigma Inc). Secondary antibodies consisted of horseradish peroxidase-conjugated goat anti-rabbit IgG or goat anti-mouse IgG (Bio-Rad, Hercules, Calif.). Detection was performed by the enhanced chemiluminescence method (Pierce, Rockford, Ill.).

Transfection of siRNA oligonucleotides. Double-stranded SMARTPOOL siRNA oligonucleotides targeting c-FLIP mRNA and double-stranded firefly luciferase control siRNA (Dharmacon Research, Lafayette, Colo.) (10 nM) were transfected into cells with Lipofectamine according to the manufacturer's instructions.

Quantitative RT-PCR. The cDNAs encoding the long isoform of FLIPL and GAPDH were amplified using the following primer pairs: 5′-CCTAGGAATCTGCCTGATAATCGA-3′ (forward primer for FLIP; SEQ ID NO:1), 5′-TGGGATATACCATGCATACTGAGATG-3′ (reverse primer for FLIP; SEQ ID NO:2), 5′-GAAGGTGAAGGTCGGAGTC-3′ (forward primer for GAPDH; SEQ ID NO:3), and 5′-GAAGATGGTGATGGGATTTC-3′ (reverse primer for GAPDH; SEQ ID NO:4). Equal amounts of cDNA for each sample were added to a prepared master mix (SYBR Green PCR Master mix, Applied Biosystems, Foster City, Calif.). Real-time quantitative PCR reactions were performed on an ABI Prism 7700 sequence detection system (Applied Biosystems, Foster City, Calif., USA). The relative abundance of a transcript was represented by the threshold cycle of amplification (C_{T}), which is inversely correlated to the amount of target RNA/first strand cDNA being amplified. To normalize for equal amounts of the latter, the transcript levels of the putative housekeeping gene GAPDH were assayed. The comparative C_{T} method was calculated as per manufacturer's instructions. To normalize the C_{T} of FLIP for each sample, the following ratio was calculated: C_{T}(FLIP)/C_{T}(GAPDH). The expression level of FLIP relative to the baseline level was calculated as 2^{-ΔΔCT(FLIP)}, where ΔCT is (average FLIP C_{T}-average GAPDH C_{T}) and ΔΔC_{T} is (average ΔC_{T} untreated sample−average ACT treated sample.

Statistics. Cytotoxicity induced by compounds used in combination with conventional agents (e.g., CH-11, TRAIL, VP-16 and staurosporine) was evaluated for evidence of synergy toxicity by comparing the slopes of the dose-response curves. If the combination of the potential sensitizing compound with the conventional agent increased the slope of the dose-response curve compared to the slopes of either the sensitizer or the conventional agent alone, then the interaction was considered synergistic. If the slopes of the curves were not significantly different, it was concluded that the enhanced toxicity was additive but not synergistic. Statistical significance was defined as a p<0.01, using two-sided tests. Synergy was confirmed by performing multiple drug dose-effect calculations using the Median Effect methods as described in Chou (“The Median-Effect Principle and the Combination Index for Quantitation of Synergism and Antagonism.” In: D. C. Rideout and T. C. Chou (eds.), Synergism and Antagonism in Chemotherapy, pp. 61-102: Academic Press, Inc., 1991).

### Results

Identification of small molecule FAS sensitizers. Resistance to death receptor ligands may permit malignant cells to escape immune surveillance and limit the clinical efficacy of recombinant death receptor ligands such as TRAIL. To identify small molecules that restore sensitivity to death receptor ligands, a cell-based high throughput screen was performed using the FAS and TRAIL-resistant prostate cancer cell line PPC-1 and a commercially available 50,000 compound library. The screens were performed in 96 well plates, to which compounds were added at 7.5 μg/ml (about 25 μM), followed by agonistic anti-FAS monoclonal antibody CH-11 (100 ng/mL). Cell viability was measured 24 hours later by MTT assay. Each plate included controls of untreated cells, cells treated only with CH-11, and cells treated with a positive control compound, 2-cyano-3,12-dioxooleana-1,9-dien-28-oic acid (CDDO), previously determined to sensitize PPC-1 cells to TNF-family death receptors and ligands (Kim et al., J. Biol. Chem. 277:22320-22329, 2002). The coefficient of variation (CV) for PPC-1 cells treated with CH-11 alone was determined to be 5%, based on 90 replicate determinations. A 50% decrease in cell viability was used as a cut-off for scoring hits.

From the primary screen of 50,000 compounds, 313 reproducible hits were obtained. To determine whether any of the 313 compounds were toxic molecules as opposed to FAS-sensitizers, the 313 compounds were evaluated in secondary screens where PPC-1 cells were treated with increasing concentrations of the compounds in the presence or absence of CH-11 antibody. Through these secondary screens, nine sensitizers were identified that increased CH-11-mediated killing above the cell death produced by treatment of the cells with the compound alone (Table 1). In contrast, the remaining 304 compounds displayed toxicity as single agents, and did not potentiate CH-11-killing.

TABLE 1

| Chembridge | | |
|---|---|---|
| ID | IUPAC Name | Structure |
| 6094911 | N-[4-chloro-3-(trifluoromethyl)phenyl]-3-oxobutanamide | 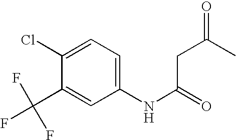 |
| 5809354 | 4-(2,4-dichlorophenoxy)-N-hydroxybutanamide | 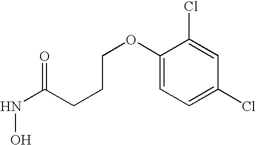 |
| 5703229 | 1-[(4-chlorophenyl)acetyl]-4-[(2e)3-phenylprop-2-enyl]piperazine | 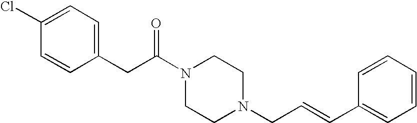 |
| 5687158 | 3,4,5-trimethoxy-N′-((5-methyltetrahydrofuran-2-yl)methyl)benzohydrazide | 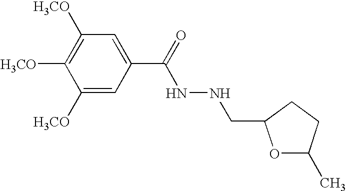 |
| 5557608 | 3,4-dimethyl-N-(3,4,5-trimethoxybenzyl)aniline | 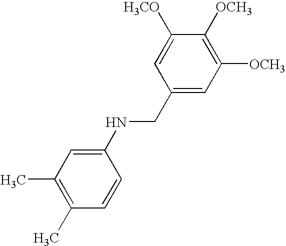 |
| 5647994 | N-(3-chloro-4-(isopentyloxy)benzyl)-imidodicarbonimidamide | 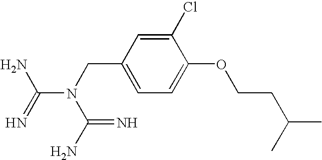 |
| 5362611 | 8-Cyclohexyl-3-methyl-5,6-dihydro-4H-pyrazino[3,2,1-jk]carbazol-3-ium; iodide | 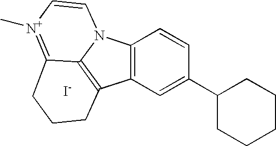 |
| 5541203 | 1-(3,5-Di-tert-butyl-4-hydroxy-phenyl)-2-(2-imino-3-methyl-2,3-dihydro-benzoimidazol-1-yl)-ethanone | 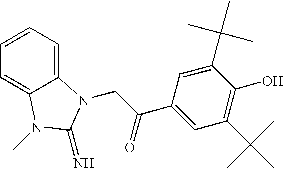 |
| 5569100 | 1-(3,5-Di-tert-butyl-4-hydroxy-phenyl)-2-(3-ethyl-2-imino-2,3-dihydro-benzoimidazol-1-yl)-ethanone | 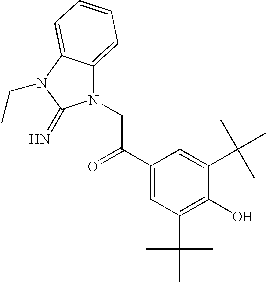 |
| 5529457 | 1-(3,5-di-tert-butyl-4-hydroxyphenyl)-2-(2-imino-3-(2-(piperidin-1-yl)ethyl)-2,3-dihydro-1H-benzo[d]imidazol-1-yl)ethanonehydrochloride | 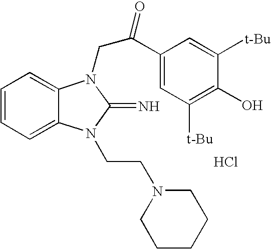 |
| 5651311 | 1-(3,5-di-tert-butyl-4-hydroxyphenyl)-2-(2-imino-3-(4-methylbenzyl)-2,3-dihydro-1H-benzo[d]imidazol-1-yl)ethanonehydrobromide | 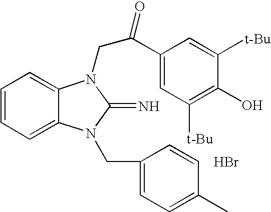 |
| 5657593 | 2-(3-benzyl-2-imino-2,3-dihydro-1H-benzo[d]imidazol-1-yl)-1-(3,5-di-tert-butyl-4-hydroxyphenyl)ethanonehydrobromide | 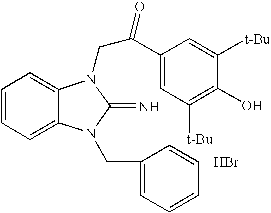 |
| 5569100 | 1-(3,5-di-tert-butyl-4-hydroxyphenyl)-2-(3-ethyl-2-imino-2,3-dihydro-1H-benzo[d]imidazol-1-yl)ethanonehydrobromide | 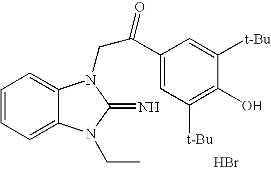 |
| 5541203 | 1-(3,5-di-tert-butyl-4-hydroxyphenyl)-2-(2-imino-3-methyl-2,3-dihydro-1H-benzo[d]imidazol-1-yl)ethanonehydrobromide | 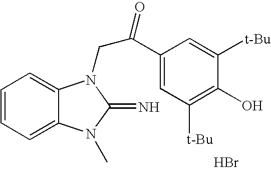 |

The identified molecules included compounds 5809354 (4-(4-chloro-2-methylphenoxy))-N-hydroxybutanamide and 6094911 (N-[4-chloro-3-(trifluoromethyl)phenyl]-3-oxobutanamide), which displayed little direct toxicity at concentrations up to 80 μM, but which sensitized PPC-1 cells to 100 ng/mL CH-11 with LD₅₀ of 20+2 μM and 35+4 μM, respectively. In contrast, other compounds such as 5703229 (1-[(4-chlorophenyl)acetyl]-4-[(2E)₃-phenylprop-2-enyl]piperazine) were directly toxic with LD₅₀ of 50+5 μM, but sensitized to CH-11 at lower concentrations with an LD₅₀ of 34+4 μM. Compounds 5809354, 6094911, and 5703229 demonstrated synergy when combined with CH-11 using the median-dose combination index (Chou, “The Median-Effect Principle and the Combination Index for Quantitation of Synergism and Antagonism.” In: D.C. Rideout and T. C. Chou (eds.), Synergism and Antagonism in Chemotherapy, pp. 61-102: Academic Press, Inc., 1991; combination Index (CI)<1). In contrast, compound 5569100 did not demonstrate synergy with this method, likely reflecting the narrow range of concentrations over which 5569100 enhanced CH-11 killing.

Effects of small molecule sensitizers on CH-11 killing of a spectrum of malignant cell lines. To assess the spectrum of activity of the small molecule FAS sensitizers, an additional nine malignant cell lines derived from breast, ovarian, and prostate carcinomas were treated with increasing concentrations of the sensitizers in the presence or absence of CH-11. Compound 6094911 sensitized four of ten tumor cell lines to CH-11, including OVCAR-3 (ovarian), T47D (breast), HT29 (colon), and PPC-1 (prostate cancer) cells. Compound 5809354 sensitized the same tumor lines with the exception of HT29. None of the other compounds sensitized more than four of ten tumor lines to CH-11. Among the non-responding tumor cell lines, for which 6094911 and 5809354 failed to sensitize, all except MDA-MB-468 expressed FAS antigen on the cell surface, as measured by flow cytometry using a specific fluorescinated antibody. Also, all of the non-responding cell lines, except Colo 205, had detectable levels of FLIP by immunoblotting. Thus, the failure of 609411 and 5809354 to sensitize these tumor lines to anti-FAS antibody was not due to either lack of FAS expression or absence of FLIP, with rare exception.

Identification of small molecules that specifically sensitize to extrinsic pathway. The nine compounds identified using anti-FAS antibody screening theoretically could non-specifically sensitize tumor cells to apoptotic stimuli or they could be selective for the extrinsic pathway. To distinguish between these two possibilities, the effects of compounds on apoptosis induced by extrinsic pathway stimuli (e.g., CH-11 antibody; TRAIL) were compared to the effects on the intrinsic pathway stimuli (e.g., etoposide [VP16]; staurosporine [STS]). Accordingly, PPC-1 cells were treated with various concentrations of compounds with or without CH-11, TRAIL, VP-16, or STS, and 24 hours later, cell viability was measured by MTT assays. Of the nine candidate compounds, eight sensitized PPC-1 cells to FAS and TRAIL (death receptor pathway stimuli) but not VP-16 or STS (intrinsic pathway stimuli), suggesting they selectively modulate the extrinsic pathway. In contrast, compound 5362611 sensitized to both the death receptor (extrinsic) and mitochondrial pathway (intrinsic) stimuli, suggesting it operates downstream at the point of convergence of these two apoptotic pathways. Similar experiments were performed for compounds 6094911, 5809354, and 5569100 using OVCAR-3 and T47D tumor lines, confirming that the results are not unique to PPC-1 cells.

Caspase-dependent induction of apoptosis by combination treatment with CH-11 and FAS-sensitizing compounds. To further assess the mechanism of the 8 compounds that selectively modulated tumor sensitivity to extrinsic pathway stimuli, the effects of benzoyl-Val-Ala-Asp-fluoromethylketone (zVAD-fmk), a broad-spectrum irreversible inhibitor of caspase-family proteases (Enzyme Systems, Dublin, Calif.) was tested. Accordingly, PPC-1 cells were treated with CH-11 and sensitizers such as 6094911, 5809354, and 5569100 with and without zVAD-fmk (100 μM) for 12 hours. Apoptosis was then measured by Annexin V staining. Consistent with a caspase-dependent mechanism of action, zVAD-fmk blocked sensitization to CH-11. Likewise, the caspase-8 inhibitory compounds acetyl-Isoleucinyl-Glutamyl-Threoninyl-Aspartyl-fluoromethylketone (Ac-IETD-fmk) (Calbiochem, San Diego, Calif.) also inhibited apoptosis induced by CH-11 in combination with these FAS-sensitizing compounds.

To further address the specificity of the compounds for the extrinsic pathway, the effects of selective apoptosis inhibitory genes were examined. For these experiments, PPC-1 cells were transfected with plasmids encoding CrmA, a viral protein that blocks the extrinsic pathway by inhibiting caspase-8 (Zhou et al., J. Biol. Chem. 272:7797-7800, 1997) or encoding Bcl-xL, a mitochondria-targeting protein that blocks the intrinsic pathway by inhibiting release of cytochrome c (Boise et al., Cell 74:597-608, 1993; Kharbanda et al., Proc. Natl. Acad. Sci. USA 94:6939-6942, 1997). After transfection, cells were treated with CH-11 antibody and compounds 6094911, 5809354, or 55619100 scoring the percentage of apoptotic cells among the successfully transfected cells as determined by co-transfection of a GFP-marker plasmid.

CrmA almost completely protected against apoptosis induced by the combination of CH-11 and either 6094911, 580935, or 5569100 while Bcl-XL had no protective effect. As a positive control, transfection of Bcl-XL but not Crm A protected PPC-1 cells from STS-induced apoptosis. Taken together, these results indicate that molecules such as 6094911, 5809354, and 5569100 specifically target the death receptor (extrinsic) pathway proximal to its convergence with the mitochondrial (intrinsic) pathway at the level of downstream effector caspases. Furthermore, since these compounds sensitize to both FAS and TRAIL, they presumably act distal to death receptors. Supporting this hypothesis, treatment of PPC-1 cells with either 6094911, 5809354 or 5569100 did not change the surface expression of FAS or TRAIL receptors, as measured by flow cytometry using specific fluorescinated antibodies.

Effects of sensitizing compounds on FLIP protein. Since FAS-sensitizing compounds 6094911 and 5809354 modulate the extrinsic pathway downstream of TNF-family death receptors but upstream of effector caspases, the effects of those compounds on expression of FLIP, an intracellular antiapoptotic protein that binds caspases-8 and -10 and is capable of suppressing death receptor signaling at a proximal point within the extrinsic pathway, was examined. For these experiments, PPC-1 cells were treated with sensitizing compounds in the presence or absence of CH-11 antibody, then cell lysates were prepared 24 hours later, and analyzed by immunoblotting for FLIP. Comparisons were made with the triterpenoid, CDDO, which was previously shown to reduce FLIP protein levels and restore sensitivity of tumor cells to FAS and TRAIL (Kim et al., J. Biol. Chem. 277:22320-22329, 2002), thus serving as a positive control.

The levels of the short isoform of FLIP were less than 5% of total FLIP protein as measured by immunoblotting and quantitative densitometry. Therefore, given the very low levels of the short isoform, the analysis was limited to the effects of the compounds on the long isoform of FLIP. Compounds 5809354 and 5569100 decreased levels of FLIP protein. Pre-treatment with z-VAD-fmk did not prevent reductions in FLIP protein, indicating that the changes in FLIP were not a secondary event mediated by caspase activation. In contrast, the other FAS-sensitizing compounds did not alter FLIP expression, indicating that they act through different mechanisms. Similar reductions in FLIP protein were observed after treating OVCAR-3, and T47D cells with 5809354. Likewise, levels of FLIP were decreased in the non-responding cell lines DU145 and MDA-MB-468 after treatment with 5809354. While these compounds reduced levels of FLIP protein, no change in levels of Bcl-2, FADD, caspase-8, or Mcl-1 were observed after treatment with these compounds.

To determine whether the reductions in FLIP protein occurred at the level of mRNA or protein, FLIP mRNA expression was measured by Quantitative RT-PCR in cells treated with 5809354 or buffer control. Treatment with 5809354 reduced expression of FLIP mRNA in the responding and non-responding cell lines. In contrast, compounds 5569100 and 6094911 did not reduce FLIP mRNA.

To determine whether the compounds activate the DISC in the presence of CH-11, processing of caspase-8 was analyzed by immunoblotting. Treatment of cells with the sensitizers and CH-11 activated caspase-8, as evidenced by a decrease in the proform of caspase-8 and an increase in the cleaved form.

To test the functional importance of decreases in FLIP by 5809354, it was determined whether knocking down FLIP could recapitulate the activity of the molecule and abrogate 5809354's ability to sensitize cells to CH-11. PPC-1 cells were transfected with double stranded siRNA that targeted FLIP or luciferase as a control. At 24 hours after transfection, cells were treated with increasing concentrations of 5809354 with or without CH-11. FLIP siRNA, but not luciferase double-strand ds RNA control, sensitized PPC-1, OVCAR-3, and T47D, but not MDA-MB-468 and DU 145 cells, to CH-11, thereby recapitulating the effects of 5809354. Furthermore, in the presence of FLIP siRNA, 5809354 the longer enhanced CH-11-mediated killing. Thus, taken together, the data suggest that decreases in FLIP by 5809354 are functionally important.

### Structure Activity Relation (SAR) Analysis.

To begin exploring the relationship between the structure and function of the FAS sensitizing compounds, a series of structurally related analogs of 6094911 were evaluated. PPC-1 cells were treated with increasing concentrations of the analogues, with and without CH-11 (100 ng/mL), and cell viability was measured by a MTT assay 24 hours later, and approximate LD₅₀ values determined (Table 2, LD₅₀ is the concentration of compound that reduces cell viability by 50%; the chloride-substituted 3-trifluor-methyl-phenyl-amide represents the core pharmacore responsible for FAS-sensitizing activity of 6094911). Comparison of the analogs indicated that modifications of the 4-chloro-3-(trifluoromethyl)-phenyl group abolished or significantly reduced activity of the compounds. Conversely, various substituents are well tolerated in the R position (Table 2). These results suggest that the N-[4-chloro-3-(trifluoromethyl)-phenyl]-amide moiety contains a pharmacophore for the activity of 6094911, while the methyl-keto moiety is expendable (Table 2).

TABLE 2

| | | LD₅₀ | |
|---|---|---|---|
| | | (μM) | LD₅₀ |
| | | (PPC1, | (μM) |
| Name | Structure | CH-11) | (PPC1) |
| N-[4-chloro-3-(trifluoromethyl)phenyl]-3-oxobutanamide | 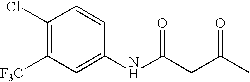 | 35 ± 4 | >100 |
| N-[4-chloro-3-(trifluoromethyl)-phenyl]-3-phenyl-proionamide | 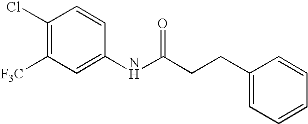 | 11 ± 1 | 85 ± 3 |
| N-(4-Chloro-3-trifluoromethyl-phenyl)-3-[(pyrazine-2-carbonyl)-hydrazono]-butyramide | 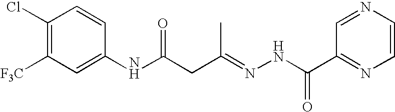 | 11 ± 3 | 67 ± 3 |
| N-[4-chloro-3-(trifluoromethyl)phenyl]-2-thiophenecar-boxamide | 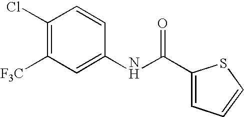 | 35 ± 11 | 96 ± 1 |
| N-[2-chloro-5-(trifluoromethyl)phenyl]-3-oxobutanamide | 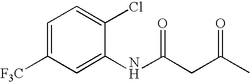 | 65 ± 5 | 75 ± 1 |
| N-[2-chloro-5-(trifluoromethyl)phenyl]-3-phenylpropan-amide | 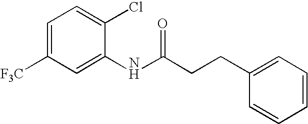 | >100 | >100 |
| N-[4-chloro-2-(trifluoromethyl)phenyl]-3-phenylpropan-amide | 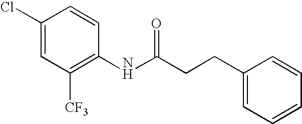 | >100 | >100 |
| 1-(3,5-di-tert-butyl-4-hydroxyphenyl)-2-(2-imino-3-(2-(piperidin-1-yl)ethyl)-2,3-dihydro-1H-benzo[d]imidazol-1-yl)ethanonehydrochloride | 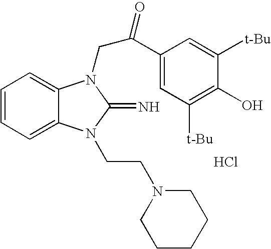 | 3.7 | 7.5 |
| 1-(3,5-di-tert-butyl-4-hydroxyphenyl)-2-(2-imino-3-(4-methylbenzyl)-2,3-dihydro-1H-benzo[d]imidazol-1-yl)ethanonehydrobromide | 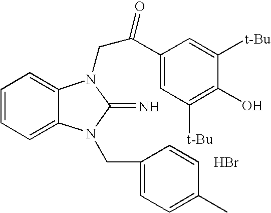 | 9 | 19 |
| 2-(3-benzyl-2-imino-2,3-dihydro-1H-benzo[d]imidazol-1-yl)-1-(3,5-di-tert-butyl-4-hydroxyphenyl)ethanonehydrobromide | 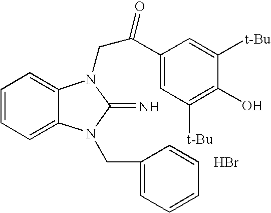 | 3.4 | 9.1 |
| 1-(3,5-di-tert-butyl-4-hydroxyphenyl)-2-(3-ethyl-2-imino-2,3-dihydro-1H-benzo[d]imidazol-1-yl)ethanonehydrobromide | 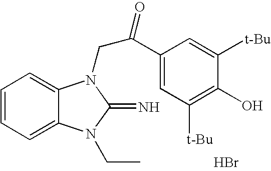 | 5.6 | 15 |
| 1-(3,5-di-tert-butyl-4-hydroxyphenyl)-2-(2-imino-3-methyl-2,3-dihydro-1H-benzo[d]imidazol-1-yl)ethanonehydrobromide | 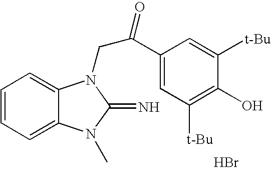 | 25.7 | 45.4 |

Among the nine active compounds originally identified, two have very similar structures, 5569100 and 5541203 (Table 1). These compounds differ only by the presence of an ethyl versus a methyl substitution on the position 3 of the dihydrobenzoimidazol group, in 5569100 and 5541203, respectively. 5569100 is approximately 5-times more potent than 5541203 as a FAS-sensitizer in PPC1 cells

(EC50 5.6 μM versus 25.7 μM) but also is three times more toxic when added alone

(without anti-FAS antibody) to cultures of PPC1 cells (LD50 15 μM versus 45 μM). Interestingly, while 5569100 reduced FLIP expression when applied to tumor cells by itself (without anti-FAS antibody), 5541203 had little effect. This observation implies that substitution of long alkyl groups to the dihydro-benzoimidazol ring may favor FLIP suppression. Additional substitutions at this position in the dihydro-benzoimidazol ring of 5569100 may provide further compounds with improved potencies.

### Discussion

Most high throughput screens are designed to identify molecules that interact with specific protein targets. In contrast, the study described here utilized a chemical biology approach to identify molecules that reverse the phenotype of FAS resistance. With the cell-based, high throughput assay, nine compounds were identified from a library of 50,000 that reversed resistance of PPC-1 cells to CH-11 anti-FAS antibody. The molecules differed in their dose-response curves with some compounds displaying FAS-independent toxicity at higher doses, while enhancing death receptor-mediated killing at lower concentrations. Some compounds such as 5934859 reversed FAS resistance only for PPC-1 cells, while other compounds such as 6094911 were more broadly acting, sensitizing 4 of 10 tumor lines to extrinsic pathway stimuli. These differences among compounds likely reflect different mechanisms of action and different cellular targets, combined with differences in FAS-resistance mechanisms among tumor cell lines. Differences in activity between cell lines may also reflect differential requirements for amplification of death receptor stimuli through the mitochondrial pathway. For example, type II cells require amplification of death receptor stimuli through the mitochondrial pathway of caspase activation to effectively induce apoptosis (Scaffidi et al., EMBO J. 17:1675-87, 1998). In these cells, blocks in the mitochondrial pathway can render these cells resistant to FAS, and thus could potentially account for resistance to the identified compounds. In type I cells, in contrast, death receptor stimuli do not require amplification through the mitochondrial pathway (Scaffidi et al., EMBO J. 17:1675-87, 1998). Of course, variations in uptake and metabolism of compounds may also contribute to the heterogeneous responses among tumor lines.

Of the nine sensitizers to CH-11 identified by this screen, eight sensitized to the extrinsic pathway agonists CH-11 and TRAIL but not to cell death stimuli that trigger the intrinsic pathway such as VP-16 and staurosporine. Furthermore, FAS-sensitization by these eight compounds was inhibited by CrmA but not Bcl-XL, consistent with a selective effect on the extrinsic pathway. These results indicate that these compounds act selectively on targets in the extrinsic pathway, operating distal to death receptors but proximal to downstream effector caspases.

Previous studies have shown that PPC-1 cell resistance to FAS and TRAIL can be reversed by decreasing the levels of the caspase-8 inhibitor FLIP using antisense oligonucleotides or the triterpenoid CDDO, which reduces FLIP expression (Kim et al., J. Biol. Chem. 277:22320-22329, 2002). Therefore, the effects of some of the compounds on levels of FLIP protein was assessed. Two of the compounds identified decreased levels of FLIP protein. The decrease in FLIP was not secondary to caspase activation, based on experiments using broad-spectrum caspase inhibitor zVAD-fmk. Reducing expression of endogenous FLIP using siRNA-based gene silencing recapitulated the ability of 5809354 to sensitize tumor cells to CH-11 and abrogated the ability of 5809354 to sensitize tumors further to CH-11. These data argue that FLIP is an important target of this compound, since the absence of the target nullifies the actions of 5809354 with respect to FAS-sensitization. 5809354 decreased FLIP mRNA and thus appears to act through a mechanism different than CDDO, which reduces FLIP protein by promoting its ubiquitination (Kim et al., J. Biol. Chem. 277:22320-22329, 2002). Of note, other extrinsic pathway modulating compounds such as 6094911 did not alter levels of FLIP protein, indicating that they act through different mechanisms.

A preliminary analysis was conducted to determine functional groups important for the activity of one of the compounds, 6094911, which enhanced FAS-induced killing of several tumor cell lines. For compound 6094911, the N-[4-chloro-3-(trifluoromethyl)-phenyl]-amide moiety appeared necessary for activity. Moving the trifluoromethyl group from the third position of the phenyl ring abolished activity of an active analog, as did moving the chloro atom from the fourth position. In contrast, a variety of substitutions are well tolerated at the R group suggesting that this position is not critical for the FAS-sensitizing activity of the compounds. Accordingly, the compounds could be derivatized at this position with affinity labels (i.e., biotin) that could results very useful to identify cellular targets of 6094911. Furthermore, the initial SAR data reported here provide some guidance for the design and synthesis of second generation compounds that may be more potent than the parent and potentially more amenable to clinical use.

In summary, compounds were identified that sensitize a spectrum of resistant cancer cells to death receptor ligand stimulation. These compounds may serve as prototypes for development of novel therapeutic adjuncts for the treatment of malignancy based on immune-based treatments such as recombinant TRAIL, agonistic anti-TRAIL antibodies, or tumor vaccines. In addition, these compounds provide new research tools for chemical biological experiments aimed at understanding mechanisms of resistance to TNF-family death ligands and death receptors.

### EXAMPLE 2

### Experimental Methods

Reagents: The 2,000-compound Spectrum Collection chemical library consisting of biologically active compounds from a collection of natural products, known drugs, and experimental biological active compounds was obtained from Microsource Discovery Systems, Inc. (Gaylordsville, Conn.). The anti-FAS monoclonal antibody CH-11 was purchased from MBL (MBL, Co. Ltd., Nagoya, Japan). TRAIL was obtained from Biomol (Plymouth Meeting, Pa.). VP-16 and Staurosporine were purchased from Sigma (Sigma Inc., Milwaukee, Wis.). 2-cyano-3,12-dioxooleana-1,9-dien-28-oic acid (CDDO) was synthesized as described (Honda et al., Bioorg Med Chem. Lett. 8: 2711-2714, 1998).

Cell Lines: Cell lines were maintained in RPMI 1640 supplemented with 2.5-10% fetal calf serum (FCS) (Hyclone, Tulare, Calif.), 1 mM L-glutamine and antibiotics (streptomycin/penicillin). Cells were cultured at 37° C. in a humidified atmosphere with 5% CO₂.

High throughput screening: Screens were performed using a fully integrated, programmable robotic liquid handling system (Biomek® FX, Beckman-Coulter Inc., Fullerton, Calif.), with integrated plate reader (LJL analyst HT 96-384, Sunnyvale, Calif.) and environmentally controlled plate carousel set at 37° C. and 5% CO₂/95% air. PPC-1 cells (1×10⁴) were seeded overnight into 96-well, flat-bottom plates (Costar, Cambridge, Mass.) in 100 μL of medium containing 2.5% FCS. The next day, aliquots from the 2,000 compound Spectrum Collection library were added at a final concentration of 25 μM in a final concentration of 0.5% (v:v) dimethylsulfoxide (DMSO). CH-11 antibody (100 ng/mL) was then added, and the cells were incubated for 24 hrs before assessing cell viability by 3-(4,5-Dimethylthiazol-2-yl)-2,5-diphenyltetrazolium Bromide (MTT) dye reduction assay (Sigma).

Cell death assays: Cell viability was measured by MTT and MTS assays, essentially as previously described (Schimmer, Cancer Cell 5:25-35, 2004). Absorbance readings were plotted against a standard curve to derive the corresponding cell number and cell viability was expressed as a percentage relative to untreated cells.

Immunoblot analysis: Protein extracts were obtained by washing cells with phosphate-buffered saline (PBS) (pH 7.4) and suspending in lysis buffer (10 mM Tris [pH 7.4], 150 mM NaCl, 0.1% Triton X-100, 0.5% sodium deoxycholate, and 5 mM EDTA) containing protease inhibitors (Complete tablets; Roche, Indianapolis, Ind.). Immunoblot assays were performed as described previously (Carter et al., Blood 105:4043-50, 2005). Briefly, equal amounts of protein as determined by Bradford assay (Bradford, Anal. Biochem. 72:248-54, 1976) were subjected to SDS-PAGE (4-20% gradient gels from ISC BioExpress, Kaysville, Utah), followed by transfer to nitrocellulose membranes. Membranes were incubated with mouse monoclonal anti-human FLIP (NF6 clone) (1:500 v/v) (Alexis, San Diego, Calif.), 1:1,000 (v/v) anti-caspase-8 clone 5F7 (Upstate) and mouse monoclonal anti-tubulin (1:2000 v/v) (Sigma Inc). Secondary antibodies consisted of horseradish peroxidase-conjugated goat anti-rabbit IgG or goat anti-mouse IgG (Bio-Rad, Hercules, Calif.). Detection was performed by the enhanced chemiluminescence method (Pierce, Rockford, Ill.).

### Results

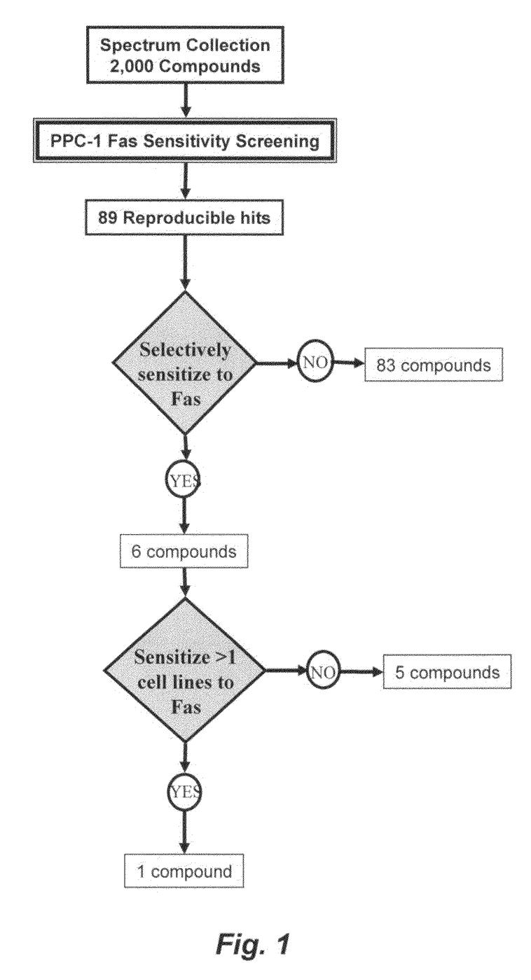

Screening of a library of natural products identifies Cyclosporine A as Fas-sensitizer. The Spectrum Collection (Microsource Discovery Systems, Inc.) of 2,000 natural products library was screened using PPC-1 cells and agonistic Fas antibody (CH-11) as described above. Of the 2,000 compounds screened, 89 produced a reduction in cell viability>50% as determined by the MTS assay. Of these 89 hits, six compounds selectively sensitized PPC-1 cells to Fas at 2.5, 5, 10, and 25 uM and did not kill PPC-1 cells when applied without anti-Fas antibody. These six compounds were then tested for ability to sensitize other tumor cell lines besides PPC-1 to anti-Fas antibody. Of these six compounds, only Cyclosporin A (CsA) displayed an ability to sensitize multiple tumor cell lines. A summary of the screening results is provided in FIG. 1.

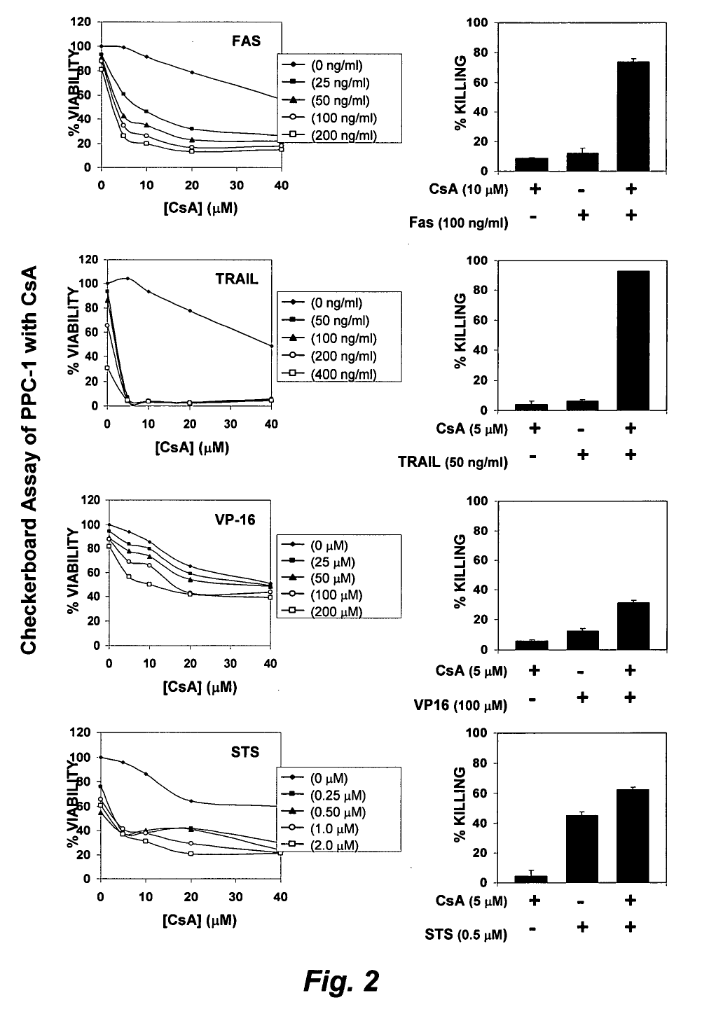

Cyclosporine A (CsA) increases sensitivity of tumor cells to stimuli of the extrinsic but not intrinsic apoptosis pathway. FIG. 2 shows that CsA sensitizes tumor cells to apoptosis induction by extrinsic pathway stimuli (agonistic anti-Fas antibody and TNF-family ligand TRAIL) but not intrinsic pathway stimuli VP16 (topoisomerase inhibitor) and Staurosporine (STS). PPC-1 prostate cancer cells (1×10⁴) were seeded overnight in 96-well plates. Cells were then treated overnight with varying concentrations of Cyclosporin A (0, 0.5, 1, 2, and 4 uM) in combination with varying concentrations of one of the following stimuli: Fas (0, 25, 50, 100, 200 ng/ml), TRAIL (0, 50, 100, 200, 400 ng/ml), VP-16 (0, 25, 50, 100, 200 uM) or STS (0, 0.25, 0.5, 1.0, and 2.0 nM). After 20-24 hours treatment, MTS was added and incubated for 4 hours. OD₄₉₀ was measured and cell viability numbers were calculated as percent of control values (mean ±SEM; n=3).

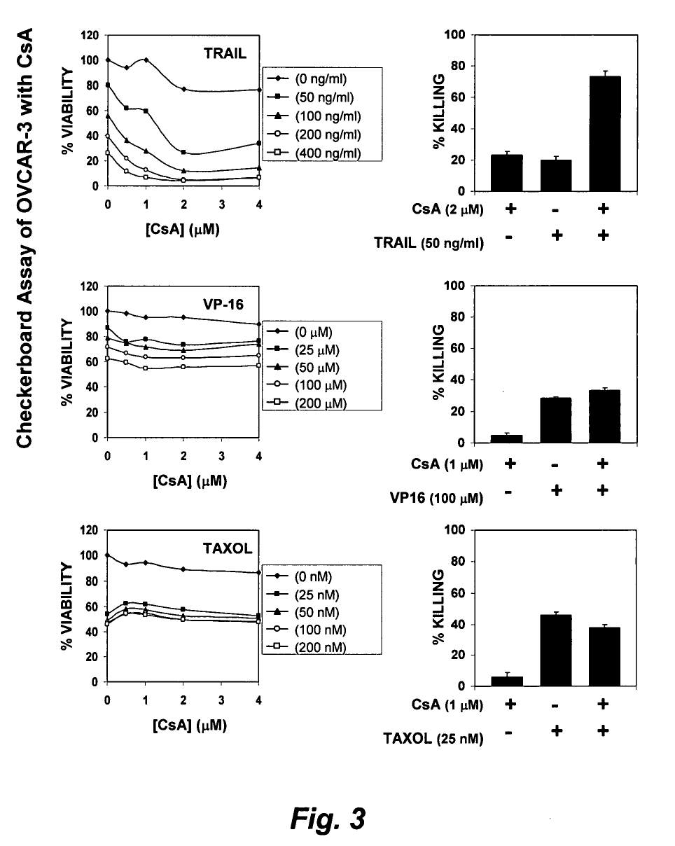

FIG. 3 shows results from similar experiment performed using OVCAR-3 ovarian cancer cells, comparing the extrinsic pathway stimulus TRAIL with intrinsic pathway stimuli, VP16 and Paclitaxel (TAXOL) (microtubule aggregator). OVCAR-3 cells (1×10⁴) were seeded overnight in 96-well plates. Cells were treated varying concentrations of Cyclosporin A (0, 0.5, 1, 2, and 4 uM) in combination with varying concentrations of one of the following stimuli: TRAIL (0, 50, 100, 200, 400 ng/ml), VP-16 (0, 25, 50, 100, 200 uM) or TAXOL (0, 25, 0.50, 100, and 200 nM). Cell viability was determined by MTS assay one day later (mean ±SEM; n=3). Concentrations of CsA and TRAIL that resulted in synergistic induction of apoptosis are shown at the right side of the figure.

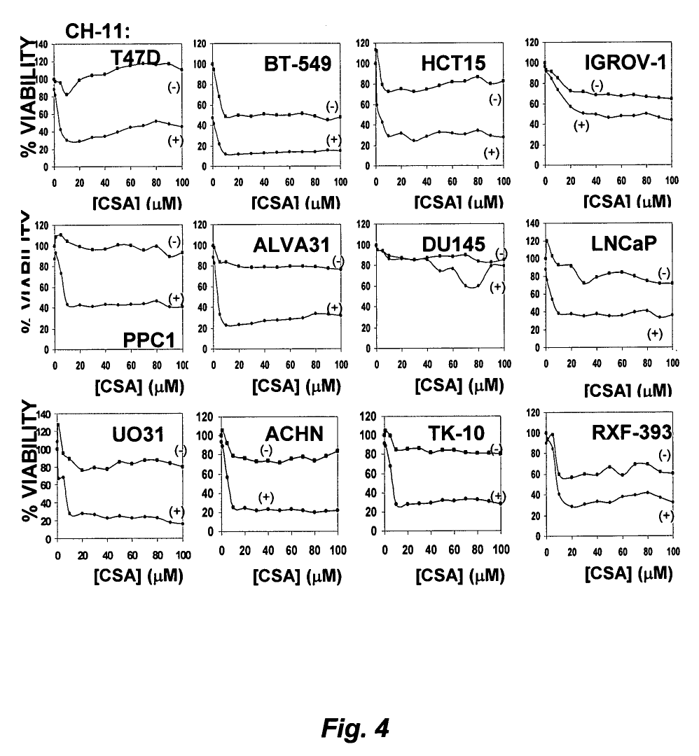

FIG. 4 shows examples of additional tumor cell lines where CsA sensitized to apoptosis induced by agonistic anti-Fas antibody CH11. Tumor cell lines included T47D and BT-549 breast cancers, HCT-15 colon cancer, IGROV-1 ovarian cancer, PPC1, ALVA-31, Du-145, and LNCaP prostate cancers, and U031, A498, 786-0, and RXF-393 renal cell cancer cell lines. Tumor cells were seeded overnight at 1×10⁴ to 5×10⁴ (based on cell type, size, and rate of growth) in RPMI, DMEM, McCoy's 5A MM, or Iscove's MEM containing 10% fetal bovine serum (FBS) with 2% Penicillin-Streptomycin in 96-well plates. Cells were then treated with increasing concentrations of Cyclosporin A (0 to 100 uM) in the presence or absence of agonistic Fas antibody (CH-11) at 100 ng/ml in the appropriate medium containing 2.5% FBS and antibiotics. After 20 to 24 hours, MTS was added and incubated for 4 hours. OD₄₉₀ measurements were taken and cell viability numbers were calculated as percent of control values.

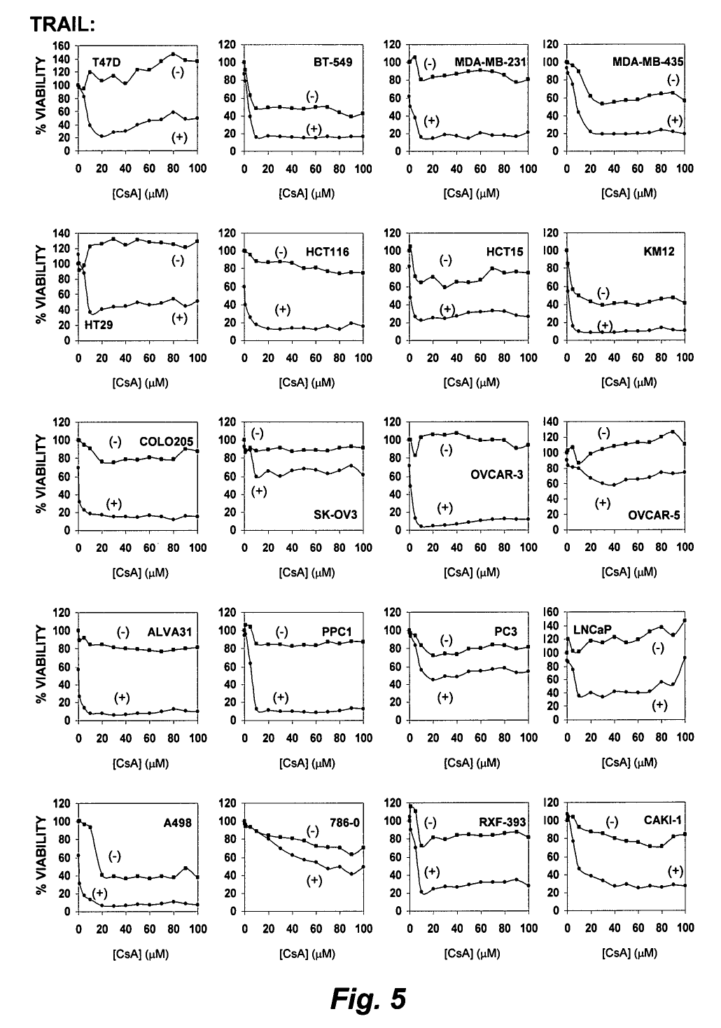

FIG. 5 shows results from a similar experiment that was performed to examine the ability of CsA to sensitize various tumor cell lines to TRAIL, a cytokine member of the TNF family that also activates the extrinsic pathway for apoptosis. Tumor cell lines, including T47D, BT-549, MDA-MB-231, and MDA-MB-435 breast cancers, HT29, HCT116, HCT15, KM12, and COL0205 colon cancers, SK-OV3, OVCAR-3, and OVCAR-5 ovarian cancers, ALVA-31, PPC-1, PC3, and LNCaP prostate cancers, and

A498, 786-0, and RXF-393 renal cell carcinoma cells, were seeded overnight at 1×10⁴ to 5×10⁴ (based on cell type, size, and rate of growth) in RPMI, DMEM, McCoy's 5A MM, or Iscove's MEM containing 10% fetal bovine serum (FBS) with 2% Penicillin-Streptomycin in 96-well plates. Tumor cells were then treated with increasing concentrations of Cyclosporine A (0 to 100 μM) in the presence or absence of recombinant TRAIL (Apo2L) at 100 ng/ml in the appropriate medium containing 2.5% FBS and antibiotics. After 20 to 24 hours, MTS was added and incubated for four hours. OD₄₉₀ measurements were performed and percentage cell viability was calculated by comparison to control untreated cells.

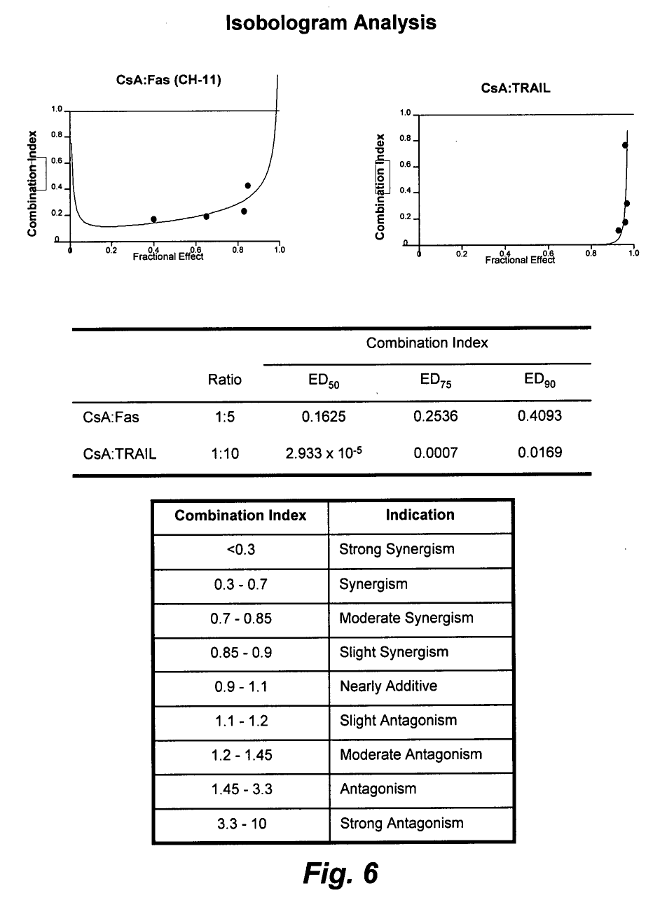

FIG. 6 shows results of isobologram analysis that was performed to mathematically explore synergy of CsA with extrinsic pathway stimuli anti-FAS and TRAIL. Using data from PPC-1 cells, where checker-board titrations were performed at fixed molar ratios of CsA: Fas or CsA:TRAIL, isobologram analysis was performed using BioSyn software. The combination index (CI) was calculated with respect to the effective dose to achieve killing of 50%, 75%, or 90% of cells. CI values<0.3 indicate strong synergism.

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed is:

1. A method of treating a cancer in a mammal in need of such treatment, comprising administrating to the mammal an amount of a composition comprising cyclosporin A that is effective to increase killing of cells of the cancer mediated by a TNF-family death receptor ligand.

2. The method of claim 1 wherein the TNF-family death receptor ligand is selected from the group consisting of TNF-α, Fas, lymphotoxin-α, TRAIL, DR3 ligand, DR6 ligand, and NGF.

3. The method of claim 2 wherein the TNF-family death receptor ligand is TRAIL.

4. The method of claim 2 wherein the TNF-family death receptor ligand is Fas.

5. The method of claim 1 wherein the TNF-family death receptor ligand is an antagonistic antibody directed against a member of the group consisting of TNFR1, FAS, DR3, DR4, DR5, DR6, and P75NTR.

6. The method of claim 1 further comprising administering to the mammal a composition comprising the TNF-family death receptor ligand.

7. The method of claim 6 comprising co-administering the composition comprising cyclosporin and the composition comprising the TNF-family death receptor ligand.

8. The method of claim 1 wherein the composition comprising cyclosporin A further comprises the TNF-family death receptor ligand.

9. The method of claim 1 further comprising administering to the mammal a composition comprising a compound of formulas (I)-(VII).

10. The method of claim 9 comprising co-administering the composition comprising cyclosporin and the composition comprising a compound of formulas (I)-(VII).

11. The method of claim 1 wherein the composition comprising cyclosporin A further comprises a compound of formulas (I)-(VII).

12. The method of claim 1 further comprising administering to the mammal a composition comprising an anti-cancer agent.

13. The method of claim 12 comprising co-administering the composition comprising cyclosporin A and the composition comprising the anti-cancer agent.

14. The method of claim 1 wherein the composition comprising cyclosporin A further comprises an anti-cancer agent.

15. The method of claim 1 wherein the cancer is selected from the group consisting of lung cancer, colon cancer, breast cancer, prostate cancer, stomach cancer, ovarian cancer, liver cancer, brain cancer, skin cancer, and blood cancer.

16. The method of claim 1 wherein the mammal is a human.

17. A method of increasing killing of cells of a cancer that is resistant to killing mediated by a TNF-family death receptor ligand, the method comprising contacting the cells ex vivo with an effective amount of a composition comprising cyclosporin A.

18. A method to enhance the efficacy of a vaccine against a cancer, comprising administering to a mammal having a cancer an effective amount of the vaccine and administering an amount of a composition comprising cyclosporin A that is effective to increase killing of cells of the cancer.

19. The method of claim 18 comprising co-administering the composition comprising cyclosporin A and the vaccine.

20. The method of claim 19 comprising administering the composition comprising cyclosporin A after administering the vaccine.

21. The method of claim 19 comprising administering the composition comprising cyclosporin A before administering the vaccine.

22. A method for identifying an agent that enhances killing of cells of a cancer that is resistant to killing mediated by a TNF-family death receptor ligand, the method comprising: (a) contacting a test sample of the cells under suitable conditions with: a composition comprising cyclosporin A; a composition comprising the agent; and a composition comprising the ligand; and (b) comparing death of the cells in the test sample to death of the cells in a control sample lacking the agent.

23. A composition for treating a cancer that is resistant to killing mediated by a TNF-family death receptor ligand, the composition comprising the ligand and an amount of cyclosporin A that is effective to increase killing of the cancer mediated by the ligand.

24. The composition of claim 23 wherein the TNF-family death receptor ligand is selected from the group consisting of TNF-α, Fas, lymphotoxin-α, TRAIL, DR3 ligand, DR6 ligand, and NGF.

25. The composition of claim 24 wherein the TNF-family death receptor ligand is TRAIL.

26. The composition of claim 24 wherein the TNF-family death receptor ligand is Fas.

27. The composition of claim 23 wherein the TNF-family death receptor ligand is an antagonistic antibody directed against a member of the group consisting of TNFR1, FAS, DR3, DR4, DR5, DR6, and P75NTR.

28. A kit for treating a cancer that is resistant to killing mediated by a TNF-family death receptor ligand, the kit comprising a composition comprising an amount of cyclosporin A that is effective to increase killing of the cancer mediated by the ligand, a composition comprising the ligand, and instructions for use.

29. A kit for treating a cancer that is resistant to killing mediated by a TNF-family death receptor ligand, the kit comprising (a) a composition comprising the ligand and an amount of cyclosporin A that is effective to increase killing of the cancer mediated by the ligand, and (b) instructions for use.
